(19) United States
(12) Patent Application Publication
(10) Pub. No.: US 2008/0038369 A1
(43) Pub. Date: Feb. 14, 2008

(54) Methods And Compositions For The Treatment Of Prolactin-Receptor Related Disorders

(76) Inventor: Ross G. Clark, Devonport (NZ)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/582,047

(22) PCT Filed: Dec. 10, 2004
(86) PCT No.: PCT/US04/41308

§ 371 Date: Apr. 21, 2007

Related U.S. Application Data
(60) Provisional application No. 60/529,235, filed on Dec. 11, 2003.
(60) Provisional application No. 60/529,252, filed on Dec. 11, 2003.

(51) Int. Cl. A61K 33/30; A61P 35/00

(52) U.S. Cl. 424/643

(57) ABSTRACT

The present invention provides methods and compositions for the treatment, diagnosis, prevention, or amelioration of one or more symptoms of a prolactin receptor-related condition, including, for example, a cancer such as breast cancer or prostate cancer, by administering a growth hormone-based prolactin receptor antagonist and zinc, or administering a growth hormone-based prolactin receptor antagonist to a tissue with an effective local concentration of zinc. The invention further provides pharmaceutical compositions of growth hormone-based prolactin receptor antagonists and zinc useful in the methods of the invention.

### 1. CROSS-REFERENCE

This application claims the benefit of U.S. Provisional Application Nos. 60/529,235, filed Dec. 11, 2003, and 60/529,252, filed Dec. 11, 2003, which applications are incorporated herein by reference in their entirety.

### 2. TECHNICAL FIELD

The present invention relates to compositions and methods for the treatment, prevention, or amelioration of one or more symptoms of conditions, disorders or diseases involving the prolactin receptor.

### 3. BACKGROUND OF THE INVENTION

More than 1.2 million Americans develop cancer each year. Cancer is the second leading cause of death in the United States, and if current trends continue, cancer is expected to be the leading cause of the death by the year 2010. Lung and prostate cancer are the most frequent cancer killers for men in the United States, while lung and breast cancer are the most frequent cancer killers for women in the United States.

Current cancer therapies include surgery, chemotherapy, hormonal therapy and/or radiation treatment to eradicate neoplastic cells in a patient (see, e.g., Stockdale, 1998, “Principles of Cancer Patient Management,” in Scientific American: Medicine, vol. 3, Rubenstein and Federman, eds., Chapter 12, Section IV). Recently, advances in cancer therapy have led to employment of biological therapy and immunotherapy to treat cancer.

Unfortunately, all known cancer treatments pose significant drawbacks for the patient. Surgery, for example, may be contraindicated due to the health of the patient or may be unacceptable to the patient. Additionally, surgery may not completely remove the neoplastic tissue. Radiation therapy is only effective when the neoplastic tissue exhibits a higher sensitivity to radiation than normal tissue, and radiation therapy can also often elicit serious side effects. Hormonal therapy is rarely given as a single agent and although it can be effective, is often used only to prevent or delay recurrence of cancer after other treatments have removed the majority of the cancer cells. Biological therapies and immunotherapies are limited in number and may produce side effects such as rashes or swellings, flu-like symptoms, including fever, chills and fatigue, digestive tract problems or allergic reactions.

Despite the availability of a variety of chemotherapeutic agents, chemotherapy has many drawbacks (see, for example, Stockdale, 1998, “Principles Of Cancer Patient Management” in Scientific American Medicine, vol. 3, Rubenstein and Federman, eds., ch. 12, sect. 10). Almost all chemotherapeutic agents are toxic, and chemotherapy causes significant, and often dangerous, side effects, such as severe nausea, bone marrow depression, and immunosuppression. Additionally, even with administration of combinations of chemotherapeutic agents, many tumor cells are resistant to or develop resistance to the chemotherapeutic agents. In fact, cells resistant to a particular chemotherapeutic agent often prove to be resistant to other drugs, even drugs that act by wholly unrelated mechanisms; this phenomenon is termed pleiotropic drug or multidrug resistance. Thus, many cancers prove refractory to standard chemotherapeutic treatment protocols because of drug resistance.

Cancer can have many causes, such as inappropriate signal transduction processes resulting in abnormal cellular proliferation. Such abnormal processes include both inappropriate receptor expression and/or inappropriate receptor/ligand interaction. For example, prolactin receptor expression in cancer tissues can be as much as 2-10 times that of normal cells (Shiu, 1979, Cancer Res. 39:4381-4386). Such increased expression has been observed in breast and prostate cancer (reviewed in Binart et al., 2000, Adv. Exp. Med. Biol. 480:85-92; Goffin et al., 1999, Genet. Anal. 15(3-5):189-201; Bonneterre et al, 1990, J. Steroid Biochem. Mol. Biol. 37(6):977-81, Blankenstein et a., 1988, Scand. J. Urol. Nephrol. Suppl. 107:39-45); intracranial tumours (see, e.g., Ciccarelli et al., 2001, J. Neurosurg. Sci. 45(2):70-4); hepatocellular carcinomas (see, e.g., Garcia-Caballero et al., 2000, Endocrine 12(3):265-71); leiomyoma and myometrial cells (see, e.g., Nowak et al., 1999, Gynecol. Obstet. Invest. 48(2):127-32); cerebral meningiomas (see, e.g., Muccioli et al., 1997, J. Endocrinol. 153(3):365-71); pituitary adenomas (see, e.g., Jin et al., 1997, J. Clin. Endocrinol. Metab. 82(3):963-8); and osteosarcoma cells (see, e.g., Bataille-Simoneau et al., 1996, Biochem. Biophys. Res. Commun. 229(1):323-8). In addition, tongue cancer cells exhibit increased local production of prolactin, while mammary cancer cells show increased local production of growth hormone (see, e.g., Bhataudekar et al., 2000, Head Neck 22:257-64 and Mol et al., 2000, Adv. Exp. Med. Biol. 480:71-6). As both of these hormones can bind the prolactin receptor, increased local production of prolactin or growth hormone can lead to prolactin receptor hyperstimulation.

In cancerous tissues where prolactin receptors are overproduced or hyperstimulated, a prolactin receptor antagonist, especially an antagonist that binds the prolactin receptor with a higher affinity than endogenous prolactin, is predicted to decrease prolactin-based signaling in cancer cells, and thus limit the proliferation of cancer cells. However, unlike growth hormone receptor antagonists, it has proved difficult to produce potent high affinity antagonists to the prolactin receptor by mutating prolactin or growth hormone (see, e.g., Kinet et al., 1999, J. Biol. Chem. 274:26033-26043). Therefore, there exists a need to devise methods to increase the affinity of ligands to the prolactin receptor, thereby producing high affinity prolactin receptor antagonists.

Accordingly, there remains a significant and unmet need for additional cancer therapies as all current treatments have significant disadvantages. Further, it is uncommon for a particular treatment to be effective to treat every instance of a given cancer. Thus, there is a need for novel therapeutic agents for the treatment of cancers and new, more effective, therapy combinations for the treatment of cancers, including prolactin-receptor related conditions such as breast cancer or prostate cancer.

Citation or discussion of a reference herein shall not be construed as an admission that such is prior art to the present invention.

### 4. SUMMARY OF THE INVENTION

The present invention provides methods and compositions for the treatment, diagnosis, prevention, or amelioration of one or more symptoms of a prolactin receptor-related condition, including, for example, a cancer such as breast cancer or prostate cancer, by administering a growth hormone-based prolactin receptor antagonist and zinc, or administering a growth hormone-based prolactin receptor antagonist to a tissue with an effective local concentration of zinc. The invention further provides pharmaceutical compositions of growth hormone-based prolactin receptor antagonists and zinc useful in the methods of the invention.

In certain aspects, the present invention relates to methods and compositions for treatment, diagnosis, prevention, or amelioration of one or more symptoms of a prolactin receptor-related condition comprising administration of a growth hormone-based prolactin receptor antagonist and zinc. In other aspects, the present invention relates to methods and compositions for treatment, diagnosis, prevention, or amelioration of one or more symptoms of conditions affected by prolactin receptor activity comprising administering a growth hormone-based prolactin receptor antagonist to a tissue with an effective local concentration of zinc.

The present invention is based, in part, on Applicant's discovery that it is possible to administer to a subject an amount of zinc sufficient to increase the affinity of growth hormone-based prolactin receptor antagonists for prolactin receptor so that binding of an administered growth hormone-based prolactin receptor antagonist decreases prolactin receptor activity in the subject.

The present invention is also based in part on the discovery that certain tissues, e.g., tumors of human breast tissue or prostate tissue, exhibit or can concentrate a local concentration of zinc that makes possible growth hormone-based prolactin receptor antagonist binding to prolactin receptor in the tissue of sufficient affinity to decrease prolactin receptor activity.

In one aspect, the invention provides methods of treating a prolactin receptor-related condition in a subject in need of such treatment, comprising administering to said subject a growth hormone-based prolactin receptor antagonist and zinc in an amount effective to treat such condition. In a representative embodiment, the growth hormone-based prolactin receptor antagonist is a human growth hormone-based antagonist. In certain embodiments, the administration of the growth hormone-based prolactin receptor antagonist and zinc can be intermittent. In other embodiments, the administration of the growth hormone-based prolactin receptor antagonist and zinc can be concurrent.

In other aspects, the invention provides methods of treating a prolactin receptor-related condition of the breast in a subject in need of such treatment, comprising administering to said subject a growth hormone-based prolactin receptor antagonist and zinc in an amount effective to treat such condition. In a representative embodiment, the growth hormone-based prolactin receptor antagonist is a human growth hormone-based antagonist. In certain embodiments, the prolactin receptor-related condition of the breast can be breast cancer. In other embodiments, the prolactin receptor-related condition of the breast can be hyperprolactinemia. In certain embodiments, the administration of the growth hormone-based prolactin receptor antagonist and zinc can be intermittent. In other embodiments, the administration of the growth hormone-based prolactin receptor antagonist and zinc can be concurrent.

In yet other aspects, the invention provides methods of treating a prolactin receptor-related condition of the prostate in a subject in need of such treatment, comprising administering to said subject a growth hormone-based prolactin receptor antagonist and zinc in an amount effective to treat such condition. In a representative embodiment, the growth hormone-based prolactin receptor antagonist is a human growth hormone-based antagonist. In certain embodiments, the prolactin receptor-related condition of the prostate can be prostate cancer. In other embodiments, the prolactin receptor-related condition of the prostate can be benign prostate hyperplasia. In certain embodiments, the administration of the growth hormone-based prolactin receptor antagonist and zinc can be intermittent. In other embodiments, the administration of the growth hormone-based prolactin receptor antagonist and zinc can be concurrent.

In another aspect, the invention provides methods of ameliorating a symptom of a prolactin receptor-related condition in a subject in need of such amelioration, comprising administering to said subject a growth hormone-based prolactin receptor antagonist and zinc in an amount effective to ameliorate such condition. In a representative embodiment, the growth hormone-based prolactin receptor antagonist is a human growth hormone-based antagonist. In certain embodiments, the administration of the growth hormone-based prolactin receptor antagonist and zinc can be intermittent. In other embodiments, the administration of the growth hormone-based prolactin receptor antagonist and zinc can be concurrent.

In another aspect, the invention provides methods of ameliorating a symptom of a prolactin receptor-related condition of the breast or prostate in a subject in need of such amelioration, comprising administering to said subject a growth hormone-based prolactin receptor antagonist and zinc in an amount effective to ameliorate such condition. In a representative embodiment, the growth hormone-based prolactin receptor antagonist is a human growth hormone-based antagonist. In certain embodiments, the administration of the growth hormone-based prolactin receptor antagonist and zinc can be intermittent. In other embodiments, the administration of the growth hormone-based prolactin receptor antagonist and zinc can be concurrent.

In other aspects, the invention provides methods of treating a prolactin receptor-related condition in a subject in need of such treatment, comprising administering to said subject a growth hormone-based prolactin receptor antagonist, wherein the growth hormone-based prolactin receptor antagonist can be administered to a tissue with an effective local concentration of zinc. Optionally, the local concentration of zinc can be further increased by additionally administering zinc to the subject. Such additional administration of zinc can be either local or systemic. In some embodiments, the tissue is breast tissue. In other embodiments, the tissue is prostate tissue.

In other aspects, the invention provides methods of treating a prolactin receptor-related condition of the breast in a subject in need of such treatment, comprising administering to said subject a growth hormone-based prolactin receptor antagonist, wherein the growth hormone-based prolactin receptor antagonist can be administered to a breast tissue with an effective local concentration of zinc. In certain embodiments, the condition of the breast is breast cancer. In other embodiments, the condition of the breast is hyperprolactinemia.

In other aspects, the invention provides methods of treating a prolactin receptor-related condition in a subject in need of such treatment, comprising administering to said subject a growth hormone-based prolactin receptor antagonist, wherein the growth hormone-based prolactin receptor antagonist can be administered to a tissue with an effective local concentration of zinc. Optionally, the local concentration of zinc can be further increased by additionally administering zinc to the subject. Such additional administration of zinc can be either local or systemic. In a representative embodiment, the tissue is prostate tissue.

In yet other aspects, the invention provides methods of treating a prolactin receptor-related condition in a subject in need of such treatment, comprising administering to said subject a growth hormone-based prolactin receptor antagonist, wherein the growth hormone-based prolactin receptor antagonist can be locally administered to a tissue with an effective local concentration of zinc, so as to target administration to such tissues. Optionally, the local concentration of zinc can be further increased by additionally administering zinc to the subject. Such additional administration of zinc can be either local or systemic. In some embodiments, the tissue is breast tissue. In other embodiments, the tissue is prostate tissue.

In yet other aspects, the invention provides methods of treating a prolactin receptor-related condition of the breast or prostate in a subject in need of such treatment, comprising administering to said subject a growth hormone-based prolactin receptor antagonist, wherein the growth hormone-based prolactin receptor antagonist can be targeted (e.g., by locally administering) to a breast tissue or prostate tissue with an effective local concentration of zinc. Optionally, the local concentration of zinc can be further increased by additionally administering zinc to the subject. Such additional administration of zinc can be either local or systemic. In certain embodiments, the condition of the breast can be breast cancer. In other embodiments, the condition of the breast is hyperprolactinemia. In certain embodiments, the condition of the prostate can be prostate cancer. In other embodiments, the condition of the prostate is benign prostate hyperplasia.

In still other aspects, the invention provides methods of preventing a prolactin receptor-related condition in a subject in need of such prevention, comprising administering to said subject a growth hormone-based prolactin receptor antagonist and zinc in an amount effective to treat such condition. In a representative embodiment, the growth hormone-based prolactin receptor antagonist is a human growth-hormone based antagonist. In certain embodiments, the administration of the growth hormone-based prolactin receptor antagonist and zinc can be intermittent. In other embodiments, the administration of the growth hormone-based prolactin receptor antagonist and zinc can be concurrent.

In certain embodiments, the invention provides methods of preventing a prolactin receptor-related condition of the breast or prostate in a subject in need of such prevention, comprising administering to said subject a growth hormone-based prolactin receptor antagonist and zinc in an amount effective to treat such condition. In a representative embodiment, the growth hormone-based prolactin receptor antagonist is a human growth-hormone based antagonist. In certain embodiments, the administration of the growth hormone-based prolactin receptor antagonist and zinc can be intermittent. In other embodiments, the administration of the growth hormone-based prolactin receptor antagonist and zinc can be concurrent.

In the methods of the invention, the growth hormone-based prolactin receptor antagonists can be any growth hormone-based prolactin receptor antagonist known to one of skill in the art without limitation. A representative growth hormone-based prolactin receptor antagonist is a human growth hormone-based prolactin receptor antagonist. Other growth hormone-based prolactin receptor antagonists described herein include, for example, growth hormone-based prolactin receptor antagonists derived from prolactin, human placental lactogen, placental growth hormone, or v-gene growth hormone.

In the methods of the invention, the zinc can be any source or form of zinc known to one of skill in the art without limitation. The zinc can be, for example, elemental zinc; zinc sulfate, ZnSO₄; zinc gluconate, Zn(C₆H₁₁O₇)₂; zinc acetate, Zn(COOCH₃)₂; or in the form of a pharmaceutically acceptable salt.

In yet other aspects, the invention further provides methods of administering a growth hormone-based prolactin receptor antagonist and zinc. The growth hormone-based prolactin receptor antagonist and zinc can be administered by any route known to one of skill in the art without limitation. In a representative embodiment, the zinc is administered orally.

In the methods of the invention, the growth hormone-based prolactin receptor antagonists and/or zinc can be formulated in pharmaceutical compositions comprising a growth hormone-based prolactin receptor antagonist, e.g., a human growth hormone-based prolactin receptor antagonist, and/or an effective amount of zinc. The pharmaceutical composition can include any physiologically acceptable carrier or excipient known to one of skill in the art without limitation.

### 4.1. Terminology

As used herein, a prolactin receptor-related condition refers to a condition affected by either systemically or locally increased prolactin concentrations or activity, or locally increased prolactin receptor number or activity. Examples of prolactin receptor-related conditions include, but are not limited to, high blood growth hormone levels (systemically or locally); prolactin-related conditions of the prostate; hyperprolactinemia; cancers such as breast tumors, colorectal tumors, prostate tumors, activated malignant B lymphocytes and lymphoma cells, promyelocyte proliferation, fibromusclar myometrial tumors (leiomyomas), and tongue cancer; and autoimmune states such as systemic lupus erythematosus, acute experimental allegic encephalomyeitis, rheumatoid arthritis, adjuvant arthritis, osteoporosis, graft v. host disease, and cystic fibrosis. A prolactin-related condition also includes cancers which on biopsy are shown to contain prolactin receptors or, in some embodiments, greater than normal expression of prolactin receptors.

A prolactin receptor-related condition of the breast refers to any breast disease or state which results in or is characterized by loss of health or integrity of the breast and includes, but is not limited to, breast cancer, e.g., mammary carcinoma, adenocarcinoma, lobular (small cell) carcinoma, intraductal carcinoma, medullary breast cancer, mucinous breast cancer, tubular breast cancer, papillary breast cancer, Paget's disease, inflammatory breast cancer, and hormone dependent tumors of the breast.

Symptoms of prolactin receptor-related conditions of the breast, particularly breast cancer, include, but are not limited to, a painless or painful lump in the breast, a lump or thickening in the breast or armpit that is new or unusual, a change in the size or shape of the breast, skin changes, such as a dimple or pucker in the skin of the breast, discharge or bleeding from the nipple that comes out without squeezing the nipple (spontaneous discharge), a nipple that in drawn inward (retraction or inversion), scaling or crusting of the nipple, change in the color or feel of the skin of the breast or the darker area around the nipple (areola), and mutation(s) in a BRCA1 or BRCA2 gene.

A prolactin receptor-related condition of the prostate refers to any prostate disease or state which results in or is characterized by loss of health or integrity of the prostate and includes, but is not limited to, prostate cancer, e.g., prostate cancer, benign prostate hyperplasia, adenocarcinoma, leiomyosarcoma, rhabdomyosarcoma, hyperprolactemia, and hormone dependent tumors of the prostate

Symptoms of prolactin receptor-related conditions of the prostate, particularly prostate cancer, include, but are not limited to, elevated levels of prostate-specific antigen (PSA) in the blood, prostate enlargement, increased urine retention, and restricted urinary flow and those described in U.S. Pat. No. 6,399,115, which is incorporated by reference in its entirety. Some of the most common symptoms of benign prostatic hypertrophy (BPH) include: a need to urinate often (especially disturbing at night), a weak or interrupted urinary stream, a feeling that one's bladder cannot empty completely, a feeling of delay or hesitation when one starts to urinate, a feeling that one must urinate right away; and continuing pain in the lower back, pelvis or upper thighs. These symptoms are caused by the way in which BPH affects the urethra and, later, the bladder. Symptoms of advanced prostate cancer include: difficulty having or keeping an erection, blood in the urine, swollen lymph nodes in the groin area, and pain in the pelvis, spine, hips, or ribs. Genetic mutations in the Hereditary Prostate Cancer Gene 1 (HPC1) or the PTEN gene, a putative protein tyrosine phosphatase gene, can also be a symptom of prostate cancer.

Zinc, as used herein, refers to any form of zinc which can safely be administered to a mammal. As used herein, zinc refers to elemental zinc, the divalent cation Zn²⁺, or any salt thereof, including but not limited to, ZnSO₄, ZnCl₂, ZnBr₂, ZnI₂, Zn(OCH₂CH₃)₂, Zn(COOCH₃)₂, and Zn(C₆H₁₁O₇)₂. In particular embodiments, the zinc salt can be zinc sulfate, ZnSO₄; zinc gluconate, Zn(C₆H₁₁O₇)₂; or in the form of any pharmaceutically acceptable salt.

As used herein, an “effective amount” of zinc refers to an amount of zinc that, when administered systemically, results in a local concentration of zinc in a tissue sufficient to increase the affinity of a growth hormone for prolactin receptor in a direct binding assay or in a competitive displacement assay relative to its affinity for the prolactin receptor in the absence of zinc.

As used herein, an “effective local concentration of zinc” refers to a concentration of zinc within a tissue in an amount to increase the affinity of a growth hormone for prolactin receptor in a direct binding assay or in a competitive displacement assay relative to its affinity for the prolactin receptor in the absence of zinc

As used herein, a tissue with an effective local concentration of zinc is a tissue that exhibits a high concentration of zinc and/or an ability to effectively concentrate zinc to a high concentration, that is, to a concentration of at least about 20 to 150 μg zinc/g blood or wet tissue. Examples of tissues with an effective local concentration of zinc include, but are not limited to, bone, brain, prostate, and mammary gland, as well as tissues that express zinc transport proteins such as the ZnT molecules (see, e.g., Cousins and McMahon, 2000, J. Nutr. 130:13845-13875) or alpha2-macroglobulin (see, e.g., Beshgetoor, 1999, J. Nutr. 129:152-7). Examples of tissues that express such transport proteins include mammary, testis, brain, and prostate tissues, as described below.

As used herein, a growth hormone-based prolactin receptor antagonist refers to a factor which neutralizes, impedes, or otherwise reduces the action or effect of a prolactin receptor. Generally, the factor is one that binds to a prolactin receptor or a growth hormone receptor with a higher affinity in the presence of zinc than in its absence. In particular, the growth hormone-based prolactin receptor antagonists described herein inhibit prolactin receptor activity and have an affinity for prolactin receptor that, in the presence of an effective concentration of zinc, is increased at least 10 fold, including at least 100 fold, such as at least 1000 fold, at least 10,000 fold, or at least 100,000 fold relative to the affinity of the growth hormone-based prolactin receptor antagonists for prolactin receptor in the absence of zinc. The affinity of the growth hormone-based prolactin receptor antagonist can be measured by ligand binding assays such as, but not limited to, direct binding assays or competitive displacement assays such as those disclosed in Section 6.2.1.1, below, and in Cunningham et al., 1990, Science 250:1709-1712, which is incorporated herein by reference in its entirety. The activity of a prolactin receptor, and the effect of a growth hormone based prolactin receptor antagonist, can be measured by assessing downstream signaling events in the prolactin signal transduction pathway, including cell proliferation, as described in Section 6.2.1.2., below, and a reference by Fuh et al., 1993, J. Biol. Chem. 268:5376-5381, which is incorporated herein by reference in its entirety. In a particular embodiment, the growth hormone-based prolactin receptor antagonist can be a polypeptide whose sequence is derived from a growth hormone polypeptide, such as a human growth hormone polypeptide, as described below.

As used herein, ameliorating a symptom refers to an improvement of at least one discernible symptom or at least one measurable physical parameter of a prolactin receptor-related condition, for example, at least one discernible symptom or at least one measurable physical parameter of cancer, such as breast or prostate cancer.

As used herein, treatment or treating refers to inhibiting the progression of a prolactin receptor-related condition, or delaying the onset of a prolactin receptor-related condition whether physically, e.g., stabilization of a discernible symptom, physiologically, e.g., stabilization of a physical parameter, or both. Treatment of cancer (e.g., breast cancer or prostate cancer) further encompasses actively intervening after onset of the disease to slow down, ameliorate symptoms of, or reverse the disease or situation. More specifically, treating, as used herein, refers to methods that treat the breast or prostate to more closely resemble that of corresponding non-diseased breast or prostate in a non-diseased state.

As used herein, prevention or preventing refers to a reduction a patient's risk of acquiring a prolactin receptor-related condition, wherein the patient has either a genetic predisposition to a prolactin receptor-related condition, such as a family history of the disease, or a non-genetic predisposition to the prolactin receptor-related condition. Prevention of breast cancer or prostate cancer also includes actively intervening as described herein prior to onset to prevent such onset of the disease.

### 5. BRIEF DESCRIPTION OF THE FIGS.

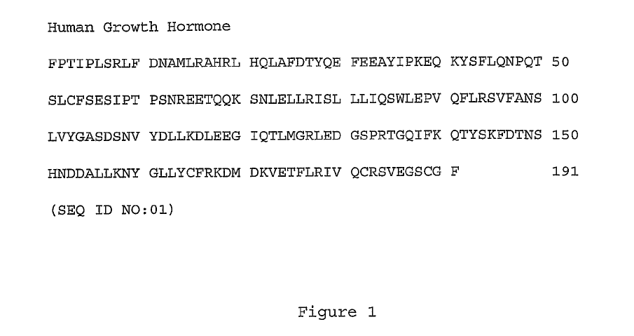

FIG. 1 presents the amino acid sequence of wild-type human growth hormone, designated SEQ ID No.: 1.

### 6. DETAILED DESCRIPTION OF THE INVENTION

The present invention relates to compositions and methods for the treatment, diagnosis, prevention, or amelioration of one or more symptoms of conditions affected by prolactin receptor activity, including, for example, cancer such as breast cancer and prostate cancer. In certain aspects, the present invention provides methods and compositions for treatment, diagnosis, prevention, or amelioration of one or more symptoms of conditions affected by prolactin receptor activity, comprising administration of a growth hormone-based prolactin receptor antagonist and zinc. In other aspects, the present invention relates to methods and compositions for treatment, diagnosis, prevention, or amelioration of one or more symptoms of conditions affected by prolactin receptor activity comprising administering a growth hormone-based prolactin receptor antagonist to a tissue with an effective local concentration of zinc (e.g., by local administration, e.g., by local injection or infusion).

These compositions and methods are based, in part, on Applicant's discovery that it is possible to administer to a subject an amount of zinc sufficient to increase the affinity of growth hormone-based prolactin receptor antagonists for prolactin receptor so that binding of an administered growth hormone-based prolactin receptor antagonist decreases prolactin receptor activity in the subject. The present invention is also based in part on the discovery that certain tissues, e.g., tumors of human breast tissue and prostate tissue, exhibit or can concentrate a local concentration of zinc adequate to yield growth hormone-based prolactin receptor antagonist binding to prolactin receptor in the tissue that is of sufficient affinity to decrease prolactin receptor activity.

### 6.1. Methods of Treating a Prolactin-Receptor Related Condition

The growth hormone-based prolactin receptor antagonists and zinc described herein, or pharmaceutically acceptable salts thereof, can be administered to a patient, such as a mammal, including a human, suffering from a prolactin receptor-related condition. Administration of one or more growth hormone-based prolactin receptor antagonist or antagonists and zinc, can be intermittent or concurrent.

In one aspect, the invention provides methods of treating a prolactin receptor-related condition in a subject in need of such treatment, comprising administering to said subject a growth hormone-based prolactin receptor antagonist and zinc in an amount effective to treat such condition. In certain embodiments, the prolactin receptor-related condition is a prolactin receptor-related condition of the breast. In further embodiments, the prolactin receptor-related condition of the breast is breast cancer. In other embodiments, the prolactin receptor-related condition of the breast can be hyperprolactinemia. In certain embodiments, the prolactin receptor-related condition is a prolactin receptor-related condition of the prostate. In further embodiments, the prolactin receptor-related condition of the prostate is prostate cancer. In other embodiments, the prolactin receptor-related condition of the prostate can be benign prostate hyperplasia.

In other aspects, invention provides methods of treating a prolactin receptor-related condition of the breast in a subject in need of such treatment, comprising administering to said subject a growth hormone-based prolactin receptor antagonist, wherein the growth hormone-based prolactin receptor antagonist is administered to breast tissue. In certain embodiments, the prolactin receptor-related condition of the breast can be breast cancer. In other embodiments, the prolactin receptor-related condition of the breast can be hyperprolactinemia.

In other aspects, invention provides methods of treating a prolactin receptor-related condition of the prostate in a subject in need of such treatment, comprising administering to said subject a growth hormone-based prolactin receptor antagonist, wherein the growth hormone-based prolactin receptor antagonist is administered to prostate tissue. In certain embodiments, the prolactin receptor-related condition of the prostate can be prostate cancer. In other embodiments, the prolactin receptor-related condition of the prostate can be benign prostate hyperplasia.

In other aspects, the invention provides methods of treating a prolactin receptor-related condition in a subject in need of such treatment, comprising administering to said subject a growth hormone-based prolactin receptor antagonist, wherein the growth hormone-based prolactin receptor antagonist can be administered to a tissue with an effective local concentration of zinc. In some embodiments, the tissue with an effective local concentration of zinc is breast tissue. In other embodiments, the tissue with an effective local concentration of zinc is prostate tissue.

In yet other aspects, the invention provides methods of treating a prolactin receptor-related condition in a subject in need of such treatment, comprising administering to said subject a growth hormone-based prolactin receptor antagonist, wherein the growth hormone-based prolactin receptor antagonist can be locally administered to a tissue with an effective local concentration of zinc.

In some embodiments, the tissue is breast tissue. In certain embodiments, the prolactin receptor-related condition is a prolactin receptor-related condition of the breast. In further embodiments, the prolactin receptor-related condition of the breast can be breast cancer. In other embodiments, the prolactin receptor-related condition of the breast can be hyperprolactinemia. Optionally, the local concentration of zinc can be further increased by additionally administering zinc to the subject. Such additional administration of zinc can be either local or systemic.

In other embodiments, the tissue is prostate tissue. In certain embodiments, the prolactin receptor-related condition is a prolactin receptor-related condition of the prostate. In further embodiments, the prolactin receptor-related condition of the prostate can be prostate cancer. In other embodiments, the prolactin receptor-related condition of the prostate can be benign prostate hyperplasia. Optionally, the local concentration of zinc can be further increased by additionally administering zinc to the subject. Such additional administration of zinc can be either local or systemic.

In certain embodiments, “treatment” or “treating” refers to an amelioration of a disease, or at least one discernible symptom thereof. In other embodiments, “treatment” or “treating” refers to an amelioration of at least one measurable physical parameter, not necessarily discernible by the patient. In yet other embodiments, “treatment” or “treating” refers to inhibiting the progression of a disease, either physically, e.g., stabilization of a discernible symptom, physiologically, e.g., stabilization of a physical parameter, or both. In still other embodiments, “treatment” or “treating” refers to delaying the onset of a disease.

In another aspect, the invention provides methods of ameliorating a symptom of a prolactin receptor-related condition in a subject in need of such amelioration, comprising administering to said subject a growth hormone-based prolactin receptor antagonist and zinc in an amount effective to ameliorate such condition. In a representative embodiment, the growth hormone-based prolactin receptor antagonist is a human growth hormone-based antagonist.

In another aspect, the invention provides methods of ameliorating a symptom of a prolactin receptor-related condition of the breast or prostate in a subject in need of such amelioration, comprising administering to said subject a growth hormone-based prolactin receptor antagonist and zinc in an amount effective to ameliorate such condition. In a representative embodiment, the growth hormone-based prolactin receptor antagonist is a human growth hormone-based antagonist.

In still other aspects, the invention provides methods of preventing a prolactin receptor-related condition in a subject in need of such prevention, comprising administering to said subject a growth hormone-based prolactin receptor antagonist and zinc in an amount effective to treat such condition. In a representative embodiment, the growth hormone-based prolactin receptor antagonist is a human growth-hormone based antagonist. In certain embodiments, the invention provides methods of preventing a prolactin receptor-related condition of the breast or prostate in a subject in need of such prevention, comprising administering to said subject a growth hormone-based prolactin receptor antagonist and zinc in an amount effective to treat such condition. In a representative embodiment, the growth hormone-based prolactin receptor antagonist is a human growth-hormone based antagonist.

In certain embodiments, the compound or a pharmaceutically acceptable salt thereof is administered to a patient, such as a mammal, including a human, as a preventative measure against cancer, such as breast cancer and prostate cancer. As used herein, “prevention” or “preventing” refers to a reduction of the risk of acquiring a disease. In certain embodiments, the compound or a pharmaceutically acceptable salt thereof is administered as a preventative measure to a patient. According to this embodiment, the patient can have a genetic predisposition to a disease, such as a family history of the disease, or a non-genetic predisposition to the disease. Accordingly, the compound and pharmaceutically acceptable salts thereof can be used for the treatment of one manifestation of a disease and prevention of another.

As described above, the invention provides methods for the treatment, prevention, or amelioration of one or more symptoms of a prolactin receptor-related condition. The prolactin receptor related condition can be any prolactin receptor related condition known by one of skill in the art to be treatable with a growth hormone-based prolactin receptor antagonist and zinc without limitation.

In certain embodiments, the prolactin receptor-related condition can be a prolactin receptor-related condition of the breast. In further embodiments, the prolactin receptor-related condition of the breast can be breast cancer. In other embodiments, the prolactin receptor-related condition of the breast can be hyperprolactinemia. In further embodiments, the prolactin receptor-related condition of the breast is selected from the group consisting of breast cancer, hyperprolactemia, mammary carcinoma, adenocarcinoma, lobular (small cell) carcinoma, intraductal carcinoma, medullary breast cancer, mucinous breast cancer, tubular breast cancer, papillary breast cancer, Paget's disease, inflammatory breast cancer, and hormone dependent tumors of the breast.

In certain embodiments, the prolactin receptor-related condition can be a prolactin receptor-related condition of the prostate. In further embodiments, the prolactin receptor-related condition of the prostate can be prostate cancer. In other embodiments, the prolactin receptor-related condition of the prostate can be benign prostate hyperplasia. In other embodiments, the prolactin receptor-related condition of the prostate is selected from the group consisting of prostate cancer, benign prostate hyperplasia, adenocarcinoma, leiomyosarcoma, rhabdomyosarcoma, hyperprolactemia, and hormone dependent tumors of the prostate.

Further, the invention also provides methods for the amelioration of one or more symptoms of a prolactin-receptor related condition, such as breast cancer and prostate cancer. Symptoms of prolactin receptor-related conditions of the breast, particularly breast cancer, that can be ameliorated according to the methods of the present invention include, but are not limited to, a painless or painful lump in the breast, a lump or thickening in the breast or armpit that is new or unusual, a change in the size or shape of the breast, skin changes, such as a dimple or pucker in the skin of the breast, discharge or bleeding from the nipple that comes out without squeezing the nipple (spontaneous discharge), a nipple that in drawn inward (retraction or inversion), scaling or crusting of the nipple, change in the color or feel of the skin of the breast or the darker area around the nipple (areola), and mutation(s) in a BRCA1 or BRCA2 gene.

Symptoms of a prolactin receptor-related condition of the prostate, i.e., prostate cancer, that can be ameliorated according to the methods of the present invention include, but are not limited to, elevated levels of prostate-specific antigen (PSA) in the blood; prostate enlargement; increased urine retention; restricted urinary flow; difficulty having or keeping an erection; blood in the urine; swollen lymph nodes in the groin area; pain in the pelvis, spine, hips, or ribs; mutations in the Hereditary Prostate Cancer Gene 1 (HPC1) or the PTEN gene; and those described in U.S. Pat. No. 6,399,115, which is incorporated by reference in its entirety. In other embodiments, symptoms of a prolactin receptor-related condition of the prostate, i.e., benign prostate hyperplasia, that can be ameliorated according to the methods of the present invention include, but are not limited to, a need to urinate often; a weak or interrupted urinary stream; a sensation that one's bladder cannot empty completely; a feeling of delay or hesitation when one starts to urinate; a feeling that one must urinate immediately; and continuing pain in the lower back, pelvis or upper thighs.

The methods of the invention further provide methods of administering a growth hormone-based prolactin receptor antagonist and zinc, as well as dosages and schedules of administration for administering growth hormone-based prolactin receptor antagonists and zinc. The methods of administration are described in Section 6.4., below, while the dosages and schedules of administration are described in Section 6.6, below.

Generally, it can be routinely determined whether a subject is suffering from or predisposed to a prolactin receptor-related condition. For example, a biopsy can be taken from a subject suspected of suffering from or predisposed to a prolactin receptor-related condition for analysis. Further, relevant tissue of a subject can be serve as a source of DNA for sequencing or subjecting to southern blot, polymerase chain reaction (“PCR”), use of the short tandem repeat (‘STR”), or restriction fragment length polymorphic (“RFLP”) analysis to determine the prolactin receptor DNA copy number in the subject. In one embodiment, the prolactin gene or growth hormone can be measured by quantitative PCR in tumors versus normal tissue (see, e.g., Touraine et al., 1998, J. Clin. Endo. Metab. 83(21):667-674).

Moreover, histological methods, for example, using antibodies or probes to detect prolactin receptors, can be used to determine if altered levels of the prolactin receptor are expressed in the subject. Biopsies from the subject can then be screened using a test for expression of the prolactin receptor. Such a test is similar to that used to detect Her-2 over-expression in breast or prostate tissue (see, e.g., Gorda et al., 2002, J Urol 168(4 Pt 1):1412-4). Subjects over-expressing the prolactin receptor represent candidates for treatment according to the methods of the invention.

In addition, it can be determined if altered levels of the prolactin receptor are expressed in the patient by western blot or other immunoassays. Such methods are well known to one of skill in the art.

### 6.2. Growth Hormone-Based Prolactin Receptor Antagonists

In the methods of the invention, growth hormone-based prolactin receptor antagonists can be used for the treatment, prevention, or amelioration of one or more symptoms of a prolactin receptor-related condition in a subject in need of such treatment, prevention, or amelioration.

A growth hormone-based prolactin receptor antagonist refers to a factor which neutralizes, impedes, or otherwise reduces the action or effect of a prolactin receptor. Generally, the factor is one that binds to a prolactin receptor or a growth hormone receptor with a higher affinity in the presence of zinc than in its absence. The growth hormone-based prolactin receptor antagonist can be any molecule that binds the prolactin receptor with a higher affinity in the presence of zinc than in its absence and inhibits prolactin receptor activity that is known to one of skill in the art without limitation. Such binding of a growth hormone-based prolactin receptor antagonist to a prolactin receptor can be assessed by assays described herein, including ligand binding assays such as, but not limited to, direct binding assays or competitive displacement assays such as those described in Section 6.2.1.1., below, and those disclosed in Cunningham et al., 1990, Science 250:1709-1712, which is incorporated herein by reference in its entirety. In addition, the activity of a prolactin receptor, and the effect of a growth hormone based prolactin receptor antagonist, can be measured by assessing downstream signaling events in the prolactin signal transduction pathway as described in Section 6.2.1.2., below. In particular, the growth hormone-based prolactin receptor antagonists described herein inhibit prolactin receptor activity and have an affinity for prolactin receptor that, in the presence of an effective concentration of zinc, is increased at least 10 fold, such as at least 100 fold, at least 1000 fold, including at least 10,000 fold, or at least 100,000 fold relative to the affinity for prolactin receptor in the absence of zinc.

Any growth hormone-based prolactin receptor antagonist can be used according to the methods of the invention. In certain embodiments, the growth hormone-based prolactin receptor antagonist can be a polypeptide whose sequence is derived from a growth hormone polypeptide, as described below. In other embodiments, the growth hormone-based prolactin receptor antagonist can be a polypeptide whose sequence is derived from human growth hormone, prolactin, human placental lactogen, placental growth hormone, or v-gene growth hormone. The methods of the invention provide for the administration of one or more growth hormone-based prolactin receptor antagonists alone or in combination. Representative growth hormone-based prolactin receptor antagonists include human growth hormone-based prolactin receptor antagonists. The sequence of native human growth hormone is presented in FIG. 1, and can also be found in Goeddel et al., 1979, Nature, 281:544-548, which is incorporated herein by reference in its entirety.

Human growth hormone-based prolactin receptor antagonists include, but are not limited to, those disclosed in Cunningham & Wells, 1991, Proc. Natl. Acad. Sci. USA 88:3407-3411; Fuh et al., 1993, J. Biol. Chem. 268(8):5376-5381; Dattani et al., 1995, J. Biol. Chem. 270(16):9222-9226; PCT Publication No. WO 94/19004; and U.S. Pat. Nos. 5,849,535; 5,958,879; 6,004,931; 6,057,292; 6,136,563, and 6,143,523; the disclosures of which are each incorporated by reference in their entireties. Examples of human growth hormone-based prolactin receptor antagonists that can be used in the methods of the invention include, but are not limited to, single mutants of human growth hormone such as F10A, N12A, L15A, R16A, E17A, R19A, D26E, F54A, S55A, E56A, S57A, P59A R64A, R64K, E65A, E66A, Q68A, Q69A, K70A, S71A, L73A, G120R, G120W, Y160A, Y164A, D171A, T175S, I179A, I179M, V180A, Q181A, R183A, S184A, V185A, E186A, G187A, S18A, double mutants of human growth hormone such as E56D/R64M or F176Y/I179T; and multiple mutants of human growth hormone such as I4A/L6A/G120A, I41/L6A/G120A/T123A, F1A/I4A/G120I/T123A, F1A/I4A/G120F, F1T/I4F/L6R/G120R/T123D, as well as any of the foregoing with an additional mutation at a residue such as R64, K172, and/or F176 that increases the affinity of site 1 for its receptor. For example, F176 is mutated to Y, and is optimally used in combination with R168N, D171SA and/or I179T, F10A, and M15W. Other multiple mutants of human growth hormone include, but are not limited to, Y11V/L113I/K115E/D116Q/E118K/E119R/G120L/Q122E/T123G/G126L/R127I/E129S, N12R/M14V/L15V/R16L/R19Y, H21A/R64K/E17A/G120R, K41R/Y42R/L45W/Q46W, K41R/Y42Q/L45W/Q46W, K41I/Y42H/L45W/Q46W, K41R/Y42R/L45W/Q46W, K41I/Y42H/L45W/Q46W/F54P/R64K, Q46H/N47D/P48S/Q49E/L52F, P48A/T50A/S51A/L52F, F54H/S55T/E56S/I58L/P59A/S62E/N63D/R64K/E66Q/T67A/K70M/S71N/N72Q/L73K/E74 D, F54P/E56D/I58T/R64K, F54P/E56W/I58T/R64K, E88G/Q91Y/F92H/R94T/S95E, and F97R/A98G/N99M/S100Q/L101D/V102A/Y103P/G104E.

In other antagonists, I4A, L6A, F1, and/or G120 can be deleted, one or more of these residues can be deleted while the remaining residues can be substituted, and/or one or more residues can be inserted adjacent to these residues. Combinations of substitutions, deletions and insertions can also be useful. One of skill in the art can optimize the activity of the growth hormone-based prolactin receptor antagonist by simply selecting particular alterations and assessing their effect in a prolactin receptor activity assay, as described in Section 6.2.1.2., below.

In representative embodiments, the human growth hormone-based prolactin receptor antagonists can be E17A, R64K, G120R, G120W, F, Y, W, D, E or I/R167N/D171S, F176Y/I179T, I4A/L6A/G120A, I41/L6A/G120A/T123A, F1A/I4A/G120I/T123A, F1A/I4A/G120F, F1T/I4F/L6R/G120R/T123D, K41R/Y42R/L45W/Q46W, K41R/Y42Q/L45W/Q46W, K41I/Y42H/L45W/Q46W, or K41I/Y42H/L45W/Q46W/F54P/R64K.

In representative embodiments, the growth hormone-based prolactin receptor antagonist has an affinity greater than that of prolactin for the prolactin receptor in the presence of zinc. Such suitable antagonists can be identified by measuring the binding affinities of the antagonists using, for example, the assays described in Section 6.2.1.1 infra.

Native human growth hormone contains two binding sites for the prolactin receptor and activates the receptor by catalyzing dimerization of two receptor molecules. In addition, the affinity of human growth hormone for the prolactin receptor is greatly enhanced by coordination of a zinc ion by three residues of the hormone, H18, H21, and G174. While mutation of one of these three residues can disrupt one of the binding sites of the human growth hormone, thereby imbuing such a mutant with antagonist activity, such mutations can also prevent coordination of zinc, preventing the mutant from binding the prolactin receptor with high affinity. Thus, human growth hormone-based prolactin receptor antagonists can include mutants of human growth hormone at one of these three residues, though such antagonists are less preferred. Examples of such human growth hormone-based prolactin receptor antagonists include the single mutants E174S and H21A, as well as the multiple mutants F1A/I4A/F10A/M14W/H18D/H21N/G120R, F, Y, W, D, E or I/R167N/D171S, F1A/I4A/H21A/R64K/E174A, I4A/G120R/E174A, I4A/G120I/E174A, F10A/M14W/H18D/H21N, F10A/M14W/H18D/H21N/R167N/D171S/E174S/F176Y/I179T, F10A/M14W/H18D/H21N/R167N/D171A/E174S/F176Y/I179T, F10A/M14W/H18D/H21N/K41I/Y42H/L45W/Q46W/F54P/R64K/R167N/D171S/E174S/F17 6Y/I179T, H18D/H21N/R167N/K168A/D171S/K172R/E174S/I179T, H18D/H21N/G120K/R167N/K168A/D171S/K172R/E174S/I179T, H18A/Q22A/F25A/D26A/Q29A/E65A/G120K/K168A/E174A, H18A/Q22A/F25A/D26A/Q29A/E65A/K168A/E174A, H18A/Q22A/F25A/D26A/Q29A/E65A/K168A/E174A, H18A/Q22A/F25A/D26A/Q29A/E65A/K168A/E174S, H18D/Q22W/F25A/D26A/Q29A/E65A/K168A/E174S, K41I/Y42H/L45W/Q46W/E174S, K41I/Y42H/L45W/Q46W/E174Y, R167D/D171S/E174S/F176Y/I179T, R167N/D171S/E174SF176Y/1179T, R167E/D171S/E174S/F176Y, and R167N/D171N/E174S/F176Y/I179T. Additional human growth hormone-based prolactin receptor antagonists include any antagonist described herein that is additionally mutated at H21 or E174, as well as the multiple mutant wherein F176 is mutated in combination with E174S and/or H18D and H21N. The prolactin receptor binding and activity assays described below can be used to assess binding of these human growth hormone-based prolactin receptor antagonists to the prolactin receptor in the presence and absence of zinc, as well as the effect of such binding on prolactin receptor activity.

In other embodiments, the growth hormone-based prolactin receptor antagonist can be a mutant of any growth hormone that corresponds to the above-described mutants of human growth hormone. One of skill in the art can align the sequences of human growth hormone and any growth hormone of known sequence by matching up conserved amino acid residues. One of skill in the art can also introduce mutations corresponding to those described above for human growth hormone in any growth hormone, thereby constructing a growth hormone-based prolactin receptor antagonist. Such growth hormone-based prolactin receptor antagonists are also within the scope of the invention.

In addition, the growth hormone-based prolactin receptor antagonists can be factors which neutralize, impede, or otherwise reduce the action or effect of a prolactin receptor and which have sequences that are derived from any of prolactin, human placental lactogen, placental growth hormone, or v-gene growth hormone, wherein the polypeptide has been mutated to allow coordination of a zinc ion. As described above, the three residues of human growth hormone that are responsible for zinc coordination have been identified. Similar residues can be introduced into prolactin receptor antagonists that are derived from prolactin, human placental lactogen, placental growth hormone, or v-gene growth hormone, thereby allowing such polypeptides to coordinate zinc. Such growth hormone-based prolactin receptor antagonists may also be administered according to the methods of the present invention.

In a representative embodiment, the human growth hormone-based prolactin receptor antagonist can also be a human growth hormone receptor antagonist, such as, but not limited to those described in Cunningham and Wells, 1991, Proc. Natl. Acad. Sci. USA 88:3407-3411 and Fuh et al., 1993, J. Biol. Chem. 268(8):5376-5381, the disclosures of which are each incorporated by reference in their entireties. A representative form of a prolactin receptor antagonist is one which binds to site 1 of both the prolactin receptor and the growth hormone receptor, but contain mutations such as, but not limited to, a G120R substitution of human growth hormone which prevents binding of the antagonist to site 2 of the prolactin receptor and the growth hormone receptor.

Furthermore, any of the above-described growth hormone-based prolactin receptor antagonists can be attached to polyethylene glycol, albumin, or some other inert compound in order to delay the clearance of the antagonist from the blood. See, e.g., U.S. Pat. Nos. 5,849,535; 6,004,931; 6,057,292; and 6,136,563 for suitable methods and compositions for attachment to growth hormone-based prolactin receptor antagonists to this end, the disclosures of which are each incorporated herein by reference in their entirety.

The growth hormone-based prolactin receptor antagonists of the present invention can be prepared by any procedure known to one of skill in the art, including, but not limited to, recombinant DNA methods, solid phase peptide synthesis techniques, or solution phase peptide synthesis techniques. The present invention encompasses sequences coding a growth hormone-based prolactin receptor antagonist, or a functionally active analogs or fragments disclosed for any species. Using known techniques of DNA recombination, the polynucleotide sequence encoding growth hormone-based prolactin receptor antagonist or a functionally active analog or fragment can be inserted into an appropriate expression vector, i.e., a vector which contains the necessary elements for the transcription and translation of the inserted protein-coding sequence. A variety of host-expression vector systems may be utilized to express the target gene coding sequences of the invention. Such host-expression systems represent vehicles by which the coding sequences of interest may be produced and subsequently purified. These include but are not limited to microorganisms such as bacteria (e.g., E. coli, B. subtilis) transformed with recombinant bacteriophage DNA, plasmid DNA or cosmid DNA expression vectors containing target gene product coding sequences; yeast (e.g., Saccharomyces, Pichia) transformed with recombinant yeast expression vectors containing the target gene product coding sequences; insect cell systems infected with recombinant virus expression vectors (e.g., baculovirus) containing the target gene product coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing target gene product coding sequences; or mammalian cell systems (e.g., COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter).

Polypeptides coding growth hormone-based prolactin receptor antagonists can be conveniently synthesized according to the usual methods of peptide chemistry, such as by solid phase peptide synthesis, as described by E. Atherton and R. C. Sheppard in “Solid Phase Peptide Synthesis” IRL Press at Oxford University Press 1989, by solution phase synthesis as described by J. Jones in “The Chemical Synthesis of Peptides”, Clarendon Press, Oxford 1994, or by both solid- and solution-phase methods, as known in the art. Polypeptides of the present invention can be purified by techniques known to those of skill in the art (e.g., preparative high performance liquid chromatography (HPLC)).

### 6.2.1. Assays for Measuring Affinity and Receptor Activity

A growth hormone-based prolactin receptor antagonist refers to a factor which neutralizes, impedes, or otherwise reduces the action or effect of a prolactin receptor. Generally, the factor is one that binds to a prolactin receptor or a growth hormone receptor with a higher affinity in the presence of zinc than in its absence. In particular, the growth hormone-based prolactin receptor antagonists described herein inhibit prolactin receptor activity and have an affinity for prolactin receptor that, in the presence of an effective concentration of zinc, is increased at least 10 fold, such as at least 100 fold, at least 1000 fold, including at least 10,000 fold, or at least 100,000 fold relative to the affinity for prolactin receptor in the absence of zinc.

The methods described herein further permit the identification of additional prolactin receptor antagonists that have increased affinity for the prolactin receptor in the presence of zinc than its absence. Such additional prolactin receptor antagonists include, but are not limited to; small molecules; synthetic drugs; peptides; polypeptides; proteins; nucleic acids (e.g., DNA and RNA nucleotides including, but not limited to, antisense nucleotide sequences, triple helices and nucleotide sequences encoding biologically active proteins, polypeptides or peptides); antibodies; synthetic or natural inorganic molecules; mimetic agents; and synthetic or natural organic molecules.

To identify an agent as a prolactin receptor antagonist that has increased affinity for the prolactin receptor in the presence of zinc than its absence, the agent can be tested for ability to antagonize the prolactin receptor using the assays for measuring prolactin receptor activity described in Section 6.2.1.2., below. The affinity for the prolactin receptor of agents that antagonize the prolactin receptor can then be assessed using the assays for measuring binding affinity described in Section 6.2.1.1., below. Agents that antagonize the prolactin receptor and exhibit increased binding affinity for the prolactin receptor in the presence of zinc as compared to their affinity in the absence of zinc are thus prolactin receptor antagonists within the scope of the invention.

### 6.2.1.1. Assays for Measuring Binding Affinity

The affinity of a growth hormone-based prolactin receptor antagonist for the prolactin receptor can be measured by any ligand binding assay known to one of skill in the art.

In one embodiment, a BIAcore™ biosensor that relies upon surface plasmon resonance to measure changes in refractive index upon hormone binding to an immobilized receptor is used to measure the affinity of the antagonist (see, e.g., U.S. Pat. No. 6,004,931, which is hereby incorporated by reference in its entirety). In another embodiment, a competitive displacement assay is used to calculate the affinity of a growth hormone-based prolactin receptor antagonist. For example, the growth hormone-based prolactin receptor antagonist is used to displace iodinated growth hormone or prolactin from the prolactin receptor (see, e.g., Fuh et al., 1993, J. Biol. Chem. 268(8):5376-5381, which is hereby incorporated by reference in its entirety).

In another embodiment, the affinity of the antagonist for the prolactin receptor can be measured by using truncated forms of the prolactin and growth hormone receptors, i.e., binding proteins of prolactin and growth hormone, respectively (see, e.g., Cunningham & Wells, 1991, Proc. Natl. Acad Sci. USA 88:3407-3411). Briefly, the binding constants for antagonists can be measured by competitive displacement of ¹²⁵I-labeled human growth hormone from a human growth hormone binding protein or a human prolactin binding protein (see, e.g., Leung et al., 1987, Nature 330:537-543; Fun et al., 1990, J. Biol. Chem. 265:3111-3115; Cunningham et al., 1990, Science 250:1709-1712; Cunningham et al., 1989, Science 243:1330-1335; and Cunningham and Wells, 1989, Science 244:1081-1085). Antibodies to the human growth hormone binding protein (see, e.g., Barnard et al., 1984, Endocrinology 115:1805-1813) or the human prolactin binding protein (see, e.g., Cunningham et al., 1990, Science 250:1709-1712) can be used to precipitate the ¹²⁵I-labeled human growth hormone:human growth hormone binding protein complex or the ¹²⁵I-labeled human growth hormone:human prolactin binding protein complex, respectively.

The affinity of the antagonist for the prolactin receptor can be quantified by calculating the relative affinity of the inhibitor, i.e., the growth hormone-based prolactin receptor antagonist, to the prolactin receptor, as compared to the affinity of prolactin or growth hormone, for the prolactin receptor by applying, for example, the Michaelis-Menten equation with respect to competitive inhibition. Such calculations are well known to one of skill in the art.

### 6.2.1.2. Assays for Measuring Receptor Activity

The activity of the prolactin receptor can be measured by the direct binding and displacement ligand binding assays described above. Prolactin receptor activity can also be measured using downstream signaling events in the prolactin signal transduction pathway, including cell proliferation.

Downstream signaling events in the prolactin signal transduction pathway include, but are not limited to, activation of Janus tyrosine kinase-2 (see, e.g., 17); phosphorylation of Stat5 proteins (see, e.g., Gouilleux et al., 1994, EMBO J 13:4361-4369 and Wakao et al., 1994, EMBO J 13:2182-2191), Stat5 activation (see, e.g., Wakao et al., 1994, EMBO J 13:2182-2191 and Kazansky et al., 1999, J Biol Chem 274:22484-22492); Stat3 activation (see, e.g., DaSilva et al., 1996, Mol Cell Endocrinol 117:131-140; Schaber et al., 1998, Cancer Res 58:1914-1919; and Yamashita et al., 1999, J Biol Chem 274:14699-14705), Stat1 activation (see, e.g., DaSilva et al., 1996, Mol Cell Endocrinol 117:131-140 and Yamashita et al., 1999, J Biol Chem 274:14699-14705), scrine/threonine kinase activation (see, e.g., Clevenger et al., 1994, J Biol Chem 269:5559-5565 and Erwin et al., 1995, Endocrinology 136:3512-3518), and protein kinase B activation (see, e.g., Hunter et al., 1997, Mol Endocrinol 11:1213-1222 and Al-Sakkaf et al., 2000, J Endocrinol 167:85-92). The activity of these signal molecules can be measured by methods known in the art, such as but not limited to immunoassays (see, e.g., Ahonen et al., 2002, Endocrinology 143(1):228-238, which is hereby incorporated by reference in its entirety). Decreased expression and/or activity of any of these signaling molecules in the presence of the antagonist indicates decreased prolactin receptor activity.

In another embodiment, prolactin receptor activity can be determined by measuring cell proliferation. Since prolactin is known to stimulate cell proliferation, the effect of a growth hormone-based prolactin receptor antagonist can be determined by measuring cell proliferation in the presence and absence of the antagonist. A decrease in cell proliferation in the presence of the antagonist, as compared to the absence of the antagonist, is indicative of decreased prolactin receptor activity. In a specific embodiment, a MTT-ESTA bioassay is used to measure increases in cell number and hormone-induced metabolic activation of individual cells (see, e.g., Dattani et al., 1995, J Biol Chem 270(16):9222-9226).

In an embodiment where the human growth hormone-based prolactin receptor antagonist is also a growth hormone receptor antagonist, the activity of the prolactin receptor can be inferred from the activity of the growth hormone receptor. In one embodiment, growth hormone receptor activity can be determined by measuring circulating levels of insulin growth factor (“IGF”), which is decreased upon growth hormone receptor inactivation (see, e.g., Trainer et al., 2000, N Engl J Med 342(16):1171-7). The present invention provides for the monitoring of a growth hormone-dependent protein from about one month to six months after administration of the growth hormone-based prolactin receptor antagonist and zinc. In a representative embodiment, the growth hormone-dependent protein is IGF-1.

In another embodiment, growth hormone receptor activity can be determined by measuring circulating levels of insulin-like growth factor binding protein-3 (“IGFBP-3”) or the acid-labile subunit of the IGFBP-3 complex, both of which are also decreased upon growth hormone receptor inactivation. See, e.g., Kopchick et al., 2002, Endo. Rev. 23(5)623-646.

### 6.3. Zinc

In the methods of the present invention, zinc can be used for the treatment, prevention, or amelioration of one or more symptoms of a prolactin receptor-related condition in a subject in need of such treatment, prevention, or amelioration.

The invention provides for administration of zinc in an amount that attains an effective concentration of zinc in tissues sufficient to increase the affinity of a growth hormone-based prolactin receptor antagonist for prolactin receptor as assayed by any ligand binding assay that measures ligand binding to prolactin receptors known in the art. In particular, the zinc concentration to be attained herein increases the affinity of a growth hormone-based prolactin receptor antagonist for prolactin receptor at least 10 fold, at least 100 fold, at least 1000 fold, including at least 10,000 fold, or at least 100,000 fold relative to the affinity for prolactin receptor in the absence of zinc.

Zinc, as used herein, refers to any form of zinc which can safely be administered to a mammal. As used herein, zinc refers to elemental zinc, the divalent cation Zn²⁺, or any salt thereof, including but not limited to, ZnSO₄, ZnCl₂, ZnBr₂, ZnI₂, Zn(OCH₂CH₃)₂, Zn(COOCH₃)₂, and Zn(C₆H₁₁O₇)₂. In particular embodiments, the zinc salt can be zinc sulfate, ZnSO₄; zinc gluconate, Zn(C₆H₁₁O₇)₂; or in the form of a pharmaceutically acceptable salt.

### 6.3.1. Assays for Measuring Zinc Concentration

In the methods of the invention, zinc concentrations can be monitored according to any method of monitoring zinc concentrations known to one of skill in the art. Such monitoring is used to determine that an effective amount of zinc is administered, the target tissue exhibits an effective local concentration of zinc, or to measure zinc concentrations after administration. In a representative embodiment, zinc concentrations are monitored for about one week to six months after administration.

Typical methods of measurement of metals in a body sample are, for example, atomic absorption spectrometry, emission spectrochemical analysis, X-ray fluorescence analysis, voltammetry, chelatometry, ultrafiltration, chromatography, and the like. In a representative embodiment, serum zinc levels are measured by atomic absorption spectroscopy (see, e.g., Taylor and Bryant, 1981, Clin. Chim, Acta 110(1):83-90 and Faure et al., 1990, Biol. Trace. Elem. Res. 24(1):25-37). In another representative embodiment, multicomponent equilibrium calculations that incorporated binding constants for zinc-transferrin as well as bicarbonate, sulfate, and phosphate binding to apotransferrin are used to model the distribution of labile zinc in normal human serum (see, e.g., Harris and Keen, 1989, J. Nutr. 119(11):1677-82). In another representative embodiment, phosphoglucomutase can be used as a metal ion indicator to measure the concentration of free zinc (see, e.g., Magneson et al., 1987, J. Biol. Chem. 262(23):11140-8). In yet another representative embodiment, non-protein bound zinc concentration in plasma can be measured by ultrafiltration (see, e.g., Bloxam et al., 1984, Clin. Chim. Acta 144(2-3):81-93). In another embodiment, an enzymatic assay is used to measure zinc levels, such as a calorimetric assay in which zinc is reacted with 2-(5-bromo-2-pyridyl)azo-5-(diethylamino)phenol in the presence of surfactants to form a complex compound. In yet another embodiment, the method described in U.S. Pat. No. 5,925,570, which is incorporated by reference in its entirety, is used to measure zinc levels in serum, blood, or a specific tissue.

Blood plasma zinc concentrations reflect zinc concentrations in tissues, although, as discussed below, much of the zinc in blood is tightly bound to blood proteins. For example, a low blood plasma zinc level is indicative of low zinc tissue concentrations (see, e.g., King et al., 2000, J. Nutr. 130:1360S-1366S). Similarly, if zinc is given, a high zinc in blood plasma will be reflective of a high tissue zinc concentration.

### 6.3.2. Bio-available Zinc

In blood, most of the zinc is tightly bound to serum proteins albumin and alpha-2-macroglobulin (see, e.g., Faure et al., 1990, Biol Trace Elem Res 24(1):25-37). This zinc is very tightly bound to these plasma proteins, and is likely not bio-available. Only about 0.2% to about 2% of the zinc in blood is loosely bound and physiologically bio-available (see, e.g., Beshgetoor & Lonhnerdal, 1999, J Nutr 129:152-157, Faure et al., 1990, Biol Trace Elem Res 24(1):25-37, Harris & Keen, 1989, J Nutr 119(11):1677-82, Magneson et al., 1987, J Biol Chem, 262(23):11140-8, Bloxam et al., 1984, Clin Chim Acta 144(2-3):81-93). These amounts of bio-available zinc are less than 0.3 mM, and possibly as little as 0.03 mM, or orders of magnitude below the 25 mM concentration shown in vitro to be necessary to maximally bind a growth hormone-based ligand to the prolactin receptor.

In contrast to blood concentrations, zinc is relatively concentrated in the prostate gland and in the mammary gland, especially in their secretions (seminal fluid and milk). In seminal plasma, there is a very high concentration of citrate, which binds zinc (see, e.g., Michalke et al., 1991, J Trace Elem Electrolytes Health Dis 5(4):251-8, Arnaud et al., 1992, J Trace Elem Electrolytes Health dis 6(2):81-90, Arver, 1982, Acta Physiol Scand 116(1):67-73, and Larue & Morfin, 1984, Endocr Res 10(2):171-81). This citrate bound zinc is loosely bound and therefore labile and bioactive. In human milk, zinc is bound almost exclusively by citrate. Therefore, the high bioavailability of zinc in the prostate and mammary gland, will favor the association of growth hormone-based ligands, such as growth hormone-based prolactin receptor antagonists.

The concentration of unbound serum zinc, protein-bound zinc, and/or zinc in tissues can be measured before and after the administration of zinc according to the methods of the present invention. Thus, these monitoring methods for determining the relative change in free zinc concentrations in serum, protein-bound zinc concentrations blood, and local zinc concentrations in tissues following zinc administration can be used in the methods of the present invention.

### 6.4. Methods of Administration

When administered to a subject, the one or more growth hormone-based prolactin receptor antagonists and/or zinc, or pharmaceutically acceptable salts thereof, can be administered as components of a composition that optionally comprises a pharmaceutically acceptable vehicle. The composition can be administered orally, or by any other convenient route, for example, by infusion or bolus injection, by absorption through epithelial or mucocutaneous linings (e.g., oral mucosa, rectal, and intestinal mucosa, etc.) and may be administered together with another biologically active agent. Administration can be systemic or local. Various delivery systems are known, e.g., encapsulation in liposomes, microparticles, microcapsules, capsules, etc., and can be used to administer the compound and pharmaceutically acceptable salts thereof.

According to the methods of the invention, the one or more growth hormone-based prolactin receptor antagonists and zinc can be administered in the same formulation or in separate formulations. Moreover, one or more growth hormone-based prolactin receptor antagonists and zinc can be administered by the same route or by different routes.

Methods of administration of the one or more growth hormone-based prolactin receptor antagonists and zinc include, but are not limited to, intradermal, intramuscular, intraperitoneal, intravenous, subcutaneous, intranasal, epidural, oral, sublingual, intranasal, intracerebral, intravaginal, transdermal, rectal, by inhalation, or topical, particularly to the ears, nose, eyes, or skin. The mode of administration is left to the discretion of the practitioner. In most instances, administration will result in the release of the compound or a pharmaceutically acceptable salt thereof into the bloodstream. In a representative embodiment, zinc is administered orally, such as a capsule or tablet. Where the one or more growth hormone-based prolactin receptor antagonists are polypeptides, the one or more growth hormone-based prolactin receptor antagonists are in some embodiments administered intravenously.

In other embodiments, the one or more growth hormone-based prolactin receptor antagonists and/or zinc, or pharmaceutically acceptable salts thereof, can be delivered in a vesicle, in particular a liposome (see Langer, 1990, Science 249:1527-1533; Treat et al., in Liposomes in the Therapy of Infectious Disease and Cancer, Lopez-Berestein and Fidler, eds., Liss, New York, pp. 353-365 (1989); Lopez-Berestein, ibid., pp. 317-327; see generally ibid.).

In yet other embodiments, the one or more growth hormone-based prolactin receptor antagonists and/or zinc, or pharmaceutically acceptable salts thereof, can be delivered in a controlled release system (see, e.g., Goodson, in Medical Applications of Controlled Release, supra, vol. 2, pp. 115-138 (1984)). Other controlled-release systems discussed in the review by Langer, 1990, Science 249:1527-1533 may be used. In certain embodiments, a pump may be used (see Langer, supra; Sefton, 1987, CRC Crit. Ref. Biomed. Eng. 14:201; Buchwald et al., 1980, Surgery 88:507; and Saudek et al., 1989, N. Engl. J. Med. 321:574). In other embodiments, polymeric materials can be used (see Medical Applications of Controlled Release, Langer and Wise, eds., CRC Pres., Boca Raton, Fla. (1974); Controlled Drug Bioavailability, Drug Product Design and Performance, Smolen and Ball, eds., Wiley, New York (1984); and Ranger and Peppas, 1983, J. Macromol. Sci. Rev. Macromol. Chem. 23:61. See also Levy et al., 1985, Science 228:190; During et al., 1989, Ann. Neurol. 25:351; and Howard et al., 1989, J. Neurosurg 71:105).

In certain embodiments, the one or more growth hormone-based prolactin receptor antagonists, or a pharmaceutically acceptable salt thereof, can be administered locally, to a tissue with an effective local concentration of zinc. Local administration may be achieved, for example, and not by way of limitation, by local infusion during surgery, topical application, e.g., in conjunction with a wound dressing after surgery, by injection, by means of a catheter, by means of a suppository, or by means of an implant, wherein the implant can be composed of a porous, non-porous, or gelatinous material, including membranes, such as sialastic membranes, or fibers. In some embodiments, the tissue with an effective local concentration of zinc can be breast tissue. In other embodiments, the tissue with an effective local concentration of zinc can be prostate tissue. In certain embodiments, the growth hormone-based prolactin receptor antagonist can be administered with an additional amount of zinc to a tissue so the resulting effective local concentration of zinc is achieved.

In other embodiments, the one or more growth hormone-based prolactin receptor antagonists can be targeted to a cancer-specific marker. Breast cancer cell-specific markers include, but are not limited to, HER2/neu (see, e.g., Dandachi et al., 2002, Lab Invest 82(8):1007-14); the sialomucin complex (see, e.g., Price-Schiavi et al., 2002, Int J Cancer 99(6):783-91); interleukin-4 receptor (see, e.g., Kawakami et al., 2001, Crit Rev Immunol. 21(1-3):299-310); and MUC1 (see, e.g., Apostolopoulos et al., 1999, Curr Opin Mol Ther 1(1):98-103).

In certain embodiments, the present invention encompasses the use of antibodies, or fragments thereof, recombinantly fused or chemically conjugated (including both covalent and non-covalent conjugations) to a growth hormone-based prolactin receptor antagonist of the present invention to generate fusion proteins. The fusion need not necessarily be direct, but may occur through linker sequences. These antibodies may be used to target the fusion protein to a particular cell type of a specific tissue (for example, breast cancer cell or prostate cancer cell) by fusing or conjugating the antibodies to antibodies specific for particular cell surface receptors. For example, such a fusion protein could be an antibody against a specific breast cancer cell marker or prostate cancer cell marker coupled to a growth hormone-based prolactin receptor antagonist. The specificity of the antibody to the cancer cell marker could target the growth hormone-based prolactin receptor antagonist to the desired cell.

Techniques for conjugating therapeutic moieties to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119-58.

In other embodiments, zinc transporter or divalent ion transporters provide additional means for targeting the human growth hormone-based prolactin receptor antagonists of the present invention. Different tissues express differing zinc transporters, such as ZnT-4 in mammary tissue, which may be associated with zinc secretion into milk; ZnT-3 in testis and brain, which may function in tissue-specific vesicular zinc transport; and ZnT-2 in prostate tissue (see, e.g., Cousins and McMahon, 2000, J. Nutr 130:1384S-1387S and Iguchi et al., 2002, J. Androl. 23(6):819-24). Targeting of the human growth hormone-based prolactin receptor antagonist to cells expressing specific zinc transporter proteins provides another method for targeting the human growth hormone-based prolactin receptor antagonists to a tissue with an effective local concentration of zinc. In addition, alpha2-macroglobulin transports zinc into mammary gland tissues (see, e.g., Beshgetoor and Lonnerdal, 1999, J. Nutr. 129(1):152-7). Thus, the alph -macroglobulin receptor provides an additional means for targeting the human growth hormone-based prolactin receptor antagonists of the present invention.

In still other embodiments, one or more growth hormone-based prolactin receptor antagonists can be cloned into a DNA vector with a tissue-specific promoter and used to transform to a specific tissue. In these embodiments, the targeted delivery of a DNA vector encoding one or more growth hormone-based prolactin receptor antagonists to a specific tissue is accomplished by methods such as, but not limited to, liposomes and virions, which are described in PCT publication WO 99/67400, the disclosure of which is incorporated by reference in its entirety.

### 6.5. Combination Therapies

In certain embodiments, subjects with cancer can be administered an effective amount of growth hormone-based prolactin receptor antagonist and zinc in combination with an additional cancer therapy.

“In combination with” as used herein refers to uses where, for example, the growth hormone-based prolactin receptor antagonist and zinc are administered during all or a portion of the entire course of administration of the additional compound; where the growth hormone-based prolactin receptor antagonist and zinc are administered for a period of time that is overlapping with the administration of the additional compound, e.g. where administration of the growth hormone-based prolactin receptor antagonist and zinc begins before the administration of the additional compound and the administration of the growth hormone-based prolactin receptor antagonist and zinc ends before the administration of the additional compound ends; where the administration of the additional compound begins before the administration of the growth hormone-based prolactin receptor antagonist and zinc and the administration of the additional compound ends before the administration of the growth hormone-based prolactin receptor antagonist and zinc ends; where the administration of the growth hormone-based prolactin receptor antagonist and zinc begins before administration of the additional compound begins and the administration of the additional compound ends before the administration of the growth hormone-based prolactin receptor antagonist and zinc ends; where the administration of the additional compound begins before administration of the growth hormone-based prolactin receptor antagonist and zinc begins and the administration of the growth hormone-based prolactin receptor antagonist and zinc ends before the administration of the additional compound ends. As such, “in combination” can also refer to regimen involving administration of two or more compounds. “In combination with” as used herein also refers to administration of two or more compounds which may be administered in the same or different formulations, by the same of different routes, and in the same or different dosage form type.

The additional cancer therapy can be any cancer therapy known to one of skill in the art without limitation. In certain embodiments, the additional cancer therapy can be one or more of chemotherapy, surgery, radiotherapy, immunotherapy, or a biological therapy.

### 6.5.1. Breast Cancer

In specific embodiments, the growth hormone receptor antagonist and zinc can be administered in combination with an effective amount of one or more other agents useful for breast cancer therapy including but not limited to: doxorubicin, epirubicin, cyclophosphamide, 5-fluorouracil, herceptin, tamoxifen, and taxanes (such as docetaxel and paclitaxel), or a combination of any or all of the foregoing agents. In a particular embodiment, one or more growth hormone receptor antagonists and zinc can be administered in combination with taxanes and standard doxorubicin and cyclophosphamide adjuvant treatment of node-positive, localized breast cancer.

In still other embodiments, other agents or methods useful for breast cancer therapy that can be used in combination with the antagonists and zinc of the present invention include, but are not limited to, mammastatin (see, e.g., U.S. Pat. No. 6,451,765), breast tumor proteins, immunogenic portions thereof, or polynucleotides that encode such portions (see, e.g., U.S. Pat. Nos. 6,432,707, 6,423,496, 6,410,507, 6,399,328, 6,387,697, 6,379,951, 6,368,796, 6,365,348, 6,344,550, 6,225,054 and 5,981,201), antiestrogen compositions (see, e.g., U.S. Pat. No. 6,306,832), selective estrogen receptor modulators (see, e.g., U.S. Pat. No. 6,300,367), cisplatin and folic acid (see, e.g., U.S. Pat. No. 6,297,245), uteroglobin (see, e.g., U.S. Pat. No. 6,288,039), edatrexate (see, e.g., U.S. Pat. No. 6,262,054), radioactive iodine (see, e.g., U.S. Pat. No. 6,238,644), the retinoid 6-[3-[1-adamantyl]-4-hydroxyphenyl]-2-naphthalene carboxylic acid (AHPN) (see, e.g., U.S. Pat. No. 6,211,239), radioactive materials (see, e.g., U.S. Pat. No. 6,179,766), interferon/antiestrogen (see, e.g., U.S. Pat. No. 6,096,301), somatostatin analogues (see, e.g., U.S. Pat. Nos. 6,066,616 and 5,753,618), polyunsaturated fatty acids, prostaglandins, prostacyclins, thromboxanes, leukotrienes, malone-aldehyde, triene epoxides (see, e.g., U.S. Pat. No. 5,766,571), inulin, oligofructose (U.S. Pat. No. 5,721,345), and Taxol (see, e.g., U.S. Pat. No. 5,496,846).

In yet other embodiments, the chemotherapy administered in combination with the one or more growth hormone receptor antagonists and zinc can include, for example, administration of a chemotherapeutic agent, e.g., cisplatin, ifosfamide, paclitaxel, taxanes, a topoisomerase I inhibitor (e.g., CPT-11, topotecan, 9-AC, or GG-211), gemcitabine, mitomycin, emetine, mitomycin, etopside, tenopside, vincristine, vinblastine, colchicin, doxordubicin, daunorubicin, dihydroxy anthracin dione, mitoxantrone, mithramycin, vinorelbine, oxaliplatin, 5-fluorouracil (5-FU), leucovorin, vinorelbine, temodal, or taxol. Such combination therapy can alternatively or additionally include radiation therapy.

In other embodiments, the one or more growth hormone-based prolactin receptor antagonists and zinc can be administered in combination with one or more agents that reduce the levels of systemic prolactin, i.e., hyperprolactemia. Such agents that reduce the levels of systemic prolactin include, but are not limited to, dopaminergic drugs, i.e., dopamine agonists, such as bromocriptine or cabergoline (see, e.g., Sabuncu et al., 2001, Intern Med 40(9):857-61).

### 6.5.2. Prostate Cancer

In other embodiments, one or more growth hormone-based prolactin receptor antagonists and zinc can be administered in combination with an effective amount of one or more other agents useful for prostate cancer therapy including but not limited to: external-beam radiation therapy, interstitial implantation of radioisotopes (i.e., I¹²⁵, palladium, iridium), leuprolide or other LHRH agonists, non-steroidal antiandrogens (flutamide, nilutamide, bicalutamide), steroidal antiandrogens (cyproterone acetate), the combination of leuprolide and flutamide, estrogens such as DES, chlorotrianisene, ethinyl estradiol, conjugated estrogens U.S.P., DES-diphosphate, radioisotopes, such as strontium-89, the combination of external-beam radiation therapy and strontium-89, second-line hormonal therapies such as aminoglutethimide, hydrocortisone, flutamide withdrawal, progesterone, and ketoconazole, low-dose prednisone, or other chemotherapy regimens reported to produce subjective improvement in symptoms and reduction in PSA level including docetaxel, paclitaxel, estramustine/docetaxel, estramustine/etoposide, estramustine/vinblastine, and estramustine/paclitaxel.

In other embodiments, other agents or methods useful for prostate cancer therapy that can be used in combination with the antagonists and zinc of the present invention include, but are not limited to, lycopene, Serenoa repens, Pygeum Africanum, and Urtica dioica, or extracts thereof (see, e.g., U.S. Pat. No. 6,399,115), a drug complex of a targeting carrier molecule, a linker acted upon by prostate specific antigen, and a drug (see, e.g., U.S. Pat. Nos. 6,391,305 and 6,368,598), brachytherapy treatment (see e.g., U.S. Pat. Nos. 6,361,487, 6,360,116, and 6,327,490), a prostate specific antigen conjugate salt (see, e.g., U.S. Pat. No. 6,355,611), a hollow suture member and a plurality of radioactive seeds located therein (see, e.g., U.S. Pat. No. 6,264,600), β-lapachone based compounds (see, e.g., U.S. Pat. No. 6,245,807), transurethral needle ablation devices (see, e.g., U.S. Pat. Nos. 6,241,702, 5,807,309, 5,762,626, 5,667,488, and 5,549,644) 2-phenyl-1-[4-(2-aminoethyl)-benzyl]-indole compounds (see, e.g., U.S. Pat. No. 6,225,308), biological agents that recognize the extracellular domain of antigens of prostate epithelial cells (see, e.g., U.S. Pat. No. 6,107,090), compounds that irreversibly bind to the androgen receptor (see, e.g., U.S. Pat. No. 6,071,957), nonsteroidal anti-androgenic compounds (see, e.g., U.S. Pat. No. 5,872,150), and 5-[(2-aminoethyl)amino]-2-[2-(diethylamino)ethyl]-2H-[1]benzothiopyrano-4,3,2-cd indazol-8-ol (see e.g., U.S. Pat. No. 5,569,667).

In yet another embodiment, combination cancer therapy can include, for example, administration of a chemotherapeutic agent, e.g., cisplatin, ifosfamide, paclitaxel, taxanes, a topoisomerase I inhibitor (e.g., CPT-11, topotecan, 9-AC, or GG-211), gemcitabine, mitomycin, emetine, mitomycin, etopside, tenopside, vincristine, vinblastine, colchicin, doxordubicin, daunorubicin, dihydroxy anthracin dione, mitoxantrone, mithramycin, vinorelbine, oxaliplatin, 5-fluorouracil (5-FU), leucovorin, vinorelbine, temodal, or taxol. Such combination cancer therapy can alternatively or additionally include, but is not limited to, radiation therapy.

In another embodiment, one or more growth hormone-based prolactin receptor antagonists and zinc can be administered in combination with one or more agents useful for prostate hypertrophy therapy useful in benign prostate hypertrophy. In a representative embodiment, one or more growth hormone-based prolactin receptor antagonists and zinc is administered in combination with an effective amount of an adrenergic receptor antagonist. In a representative embodiment, the adrenergic receptor antagonists are alpha adrenergic receptor antagonists, including alpha₁ adrenergic receptor antagonist, such as, but not limited to, quinazolines. In a representative embodiment, the quinazonline is CARDURA® (doxazosin mesylate). In another embodiment, alpha-1 receptor blockers terazosin (marketed as Hytrin®), and tamsulosin (marketed as Flomax®), can also be used in combination with the growth hormone-based prolactin receptor antagonists and zinc of the present invention. In yet another embodiment, beta3 subtype adrenergic receptor agonists and antagonists described in U.S. Pat. No. 5,627,200 can be used in combination with the growth hormone-based prolactin receptor antagonists and zinc of the present invention. In another embodiment, drugs that affect steroid metabolism, such as but not limited to androgen antagonists, such as drugs that affect steroid metabolism (e.g., Finasteride, a specific inhibitor of the intracellular enzyme which converts the androgen testosterone to 5-alpha-dihydrotestosterone), can be used in combination with the growth hormone-based prolactin receptor antagonists and zinc of the present invention.

In another embodiment, one or more growth hormone-based prolactin receptor antagonists and zinc can be administered in combination with one or more agents that reduce the levels of systemic prolactin, i.e., hyperprolactemia. Such agents that reduce the levels of systemic prolactin include, but are not limited to, dopaminergic drugs, i.e., dopamine agonists, such as bromocriptine or cabergoline (see, e.g., Sabuncu et al., 2001, Intern Med 40(9):857-61).

### 6.6. Dosage and Schedule of Administration

The methods of the invention provide for the administration of one or more growth hormone-based prolactin receptor antagonist or antagonists in combination with zinc. The combinations of any of the growth hormone-based prolactin receptor antagonists and/or forms of zinc described in Sections 6.2 and 6.3 can be administered intermittently.

Therefore, in certain embodiments of the invention, the methods for the treatment, prevention, or amelioration of one or more symptoms of a prolactin receptor-related condition encompass intermittently administering one or more growth hormone-based prolactin receptor antagonists and/or zinc. The frequency of the intermittent administration can be limited by a number of factors, including but not limited to, the pharmacokinetic parameters of the formulation and the pharmacodynamic effects of the growth hormone-based prolactin receptor antagonists and zinc on the subject.

The following is exemplary only and merely serves to illustrate that the term “intermittent” can encompass any administration regimen designed by a person of ordinary skill in the art.

In one example, the one or more growth hormone-based prolactin receptor antagonists and zinc can be administered sequentially. In such sequential administration of the one or more growth hormone-based prolactin receptor antagonist and zinc, the one or more growth hormone-based prolactin receptor antagonist can be administered first, followed by zinc, or vice versa. The sequential addition of compounds can involve two or more compounds. One skilled in the art can determine the necessary sequence of compounds to exert the desired effect. The administration can continue for one, two, three, or four weeks or one, two, three, four, five, or six months, or one year, or longer. Optionally, after a period of rest, the one or more growth hormone-based prolactin receptor antagonists and zinc can be administered under the same or different schedule. The period of rest can be one, two, three, or four weeks, or longer, according to the pharmacodynamic effects of the one or more growth hormone-based prolactin receptor antagonists and zinc on the subject.

In another example, the one or more growth hormone-based prolactin receptor antagonists and/or zinc can be administered about once per day. The administration can continue for one, two, three, or four weeks or one, two, three, four, five, or six months, or one year, or longer. Optionally, after a period of rest, the one or more growth hormone-based prolactin receptor antagonists and zinc can be administered under the same or different schedule. The period of rest can be one, two, three, or four weeks, or longer, according to the pharmacodynamic effects of the one or more growth hormone-based prolactin receptor antagonists and zinc on the subject.

In another example, the one or more growth hormone-based prolactin receptor antagonists and/or zinc can be administered about once every two days. The administration can continue for one, two, three, or four weeks or one, two, three, four, five, or six months, or one year, or longer. Optionally, after a period of rest, the one or more growth hormone-based prolactin receptor antagonists and zinc can be administered under the same or different schedule. The period of rest can be one, two, three, or four weeks, or longer, according to the pharmacodynamic effects of the one or more growth hormone-based prolactin receptor antagonists and zinc on the subject.

In another example, the one or more growth hormone-based prolactin receptor antagonists and/or zinc can be administered about once every three days. The administration can continue for one, two, three, or four weeks or one, two, three, four, five, or six months, or one year, or longer. Optionally, after a period of rest, the one or more growth hormone-based prolactin receptor antagonists and zinc can be administered under the same or different schedule. The period of rest can be one, two, three, or four weeks, or longer, according to the pharmacodynamic effects of the one or more growth hormone-based prolactin receptor antagonists and zinc on the subject.

In another example, the one or more growth hormone-based prolactin receptor antagonists and/or zinc can be administered about once every four days. The administration can continue for one, two, three, or four weeks or one, two, three, four, five, or six months, or one year, or longer. Optionally, after a period of rest, the one or more growth hormone-based prolactin receptor antagonists and zinc can be administered under the same or different schedule. The period of rest can be one, two, three, or four weeks, or longer, according to the pharmacodynamic effects of the one or more growth hormone-based prolactin receptor antagonists and zinc on the subject.

In still another example, the one or more growth hormone-based prolactin receptor antagonists and/or zinc can be administered about once every five days. The administration can continue for one, two, three, or four weeks or one, two, three, four, five, or six months, or one year, or longer. Optionally, after a period of rest, the one or more growth hormone-based prolactin receptor antagonists and zinc can be administered under the same or different schedule. The period of rest can be one, two, three, or four weeks, or longer, according to the pharmacodynamic effects of the one or more growth hormone-based prolactin receptor antagonists and zinc on the subject.

In yet another example, the one or more growth hormone-based prolactin receptor antagonists and/or zinc can be administered about once every six days. The administration can continue for one, two, three, or four weeks or one, two, three, four, five, or six months, or one year, or longer. Optionally, after a period of rest, the one or more growth hormone-based prolactin receptor antagonists and zinc can be administered under the same or different schedule. The period of rest can be one, two, three, or four weeks, or longer, according to the pharmacodynamic effects of the one or more growth hormone-based prolactin receptor antagonists and zinc on the subject.

In still another example, the one or more growth hormone-based prolactin receptor antagonists and/or zinc can be administered about once every seven days. The administration can continue for one, two, three, or four weeks or one, two, three, four, five, or six months, or one year, or longer. Optionally, after a period of rest, the one or more growth hormone-based prolactin receptor antagonists and zinc can be administered under the same or different schedule. The period of rest can be one, two, three, or four weeks, or longer, according to the pharmacodynamic effects of the one or more growth hormone-based prolactin receptor antagonists and zinc on the subject.

The above-described administration schedules are provided for illustrative purposes only and should not be considered limiting. A person of ordinary skill in the art will readily understand that all growth hormone-based prolactin receptor antagonists are within the scope of the invention; that human growth hormone-based prolactin receptor antagonists are preferred; and that the exact dosing and schedule of administration of the one or more growth hormone-based prolactin receptor antagonists and/or zinc can vary due to many factors. For example, the formulation of the one or more growth hormone-based prolactin receptor antagonists or zinc can affect the schedule of administration, as certain time-release formulations, e.g., a depot formulation, can allow less frequent administration of the antagonist or zinc than would otherwise be possible. Such formulations are described in Section 6.7, below.

The methods of the invention further provide for the concurrent administration of one or more growth hormone-based prolactin receptor antagonist or antagonists in combination with zinc. The combinations of any of the growth hormone-based prolactin receptor antagonists and/or forms of zinc described in Sections 6.2 and 6.3 can be administered concurrently.

Therefore, in certain embodiments of the invention, the methods for the treatment, prevention, or amelioration of one or more symptoms of a prolactin receptor-related condition encompass concurrently administering one or more growth hormone-based prolactin receptor antagonists and zinc. The frequency of the concurrent administration can be limited by a number of factors, including but not limited to, the pharmacokinetic parameters of the formulation and the pharmacodynamic effects of the growth hormone-based prolactin receptor antagonists and zinc on the subject. In certain of these embodiments, the one or more growth hormone-based prolactin receptor antagonist or antagonists can be administered within about 1, 5, 10, 20, 30, 40, 50, or 60 minutes, or 1, 2, 3, 4, 5, 6, 7, 8, 9, 10, 11, 12, 13, 14, 15, 16, 17, 18, 19, 20, 21, 22, or 23 hours before or after the zinc is administered.

Independently of the frequency of administration of the one or more growth hormone-based prolactin receptor antagonists, zinc can be administered at a frequency of about daily, and in a more representative embodiment, two to four times daily (see, e.g., Al-Gurairi et al., 2002, Brit. J. Dermatol. 146(3):423-431).

The amount of a therapeutically effective amount of a pharmaceutical agent in the acute or chronic management of a disease or disorder may differ depending on factors including but not limited to the disease or disorder treated, the specific pharmaceutical agents and the route of administration. According to the methods of the invention, a therapeutically effective amount of one or more growth hormone-based prolactin receptor antagonists and zinc is any amount of one or more growth hormone-based prolactin receptor antagonists and zinc effective to treat or prevent a prolactin receptor-related condition, or ameliorate one or more symptoms thereof. The dose, dose frequency, duration, or any combination, may also vary according to age, body weight, response, and the past medical history of the subject as well as the route of administration, pharmacokinetic and pharmacodynamic effects of the pharmaceutical agent. These factors are routinely considered by one of skill in the art.

In certain embodiments, the methods of the invention provide for administration of one or more growth hormone-based prolactin receptor antagonists while monitoring serum IGF-1, IGFBP-3, acid labile subunit of IGFBP, or other IGFBP concentrations. The one or more growth hormone-based prolactin receptor antagonists can be administered in increasing amounts until serum IGF-1 or IGFBP concentrations decline, including by more than 50%. This amount of growth hormone-based prolactin receptor antagonist can then be administered to a subject according to the schedules of administration described above. A zinc therapy can then commence with administration of increasing amounts of zinc, while measuring zinc concentration in blood and monitoring zinc-related side effects. Once a maximal tolerated amount of zinc is determined, this amount can then be administered to a subject according to the schedules of administration described above.

Toxicity and therapeutic efficacy of growth hormone-based prolactin receptor antagonists can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Growth hormone-based prolactin receptor antagonists which exhibit large therapeutic indices are preferred. While growth hormone-based prolactin receptor antagonists that exhibit toxic side effects may be used, care should be taken to design a delivery system that targets such antagonists to the site of affected tissue in order to minimize potential damage to uninfected cells and, thereby, reduce side effects.

The data obtained from the cell culture assays and animal studies can be used in formulating a range of dosage of growth hormone-based prolactin receptor antagonists for use in humans. The dosage of such antagonists lies within a range of circulating concentrations that include the ED₅₀ with little or no toxicity. The dosage may vary within this range depending upon the dosage form employed and the route of administration utilized. For any growth hormone-based prolactin receptor antagonist used in the method of the invention, the therapeutically effective amount can be estimated initially from cell culture assays. A dose may be formulated in animal models to achieve a circulating plasma concentration range that includes the IC₅₀ (i.e., the concentration of the test antagonist which achieves a half-maximal inhibition of symptoms) as determined in cell culture. Such information can be used to more accurately determine useful doses in humans. Plasma concentrations may be measured, for example, by high performance liquid chromatography.

In certain embodiments, a therapeutically effective amount of a growth hormone-based prolactin receptor antagonist can range from about 0.001 mg/kg body weight to 500 mg/kg body weight, such as about 0.01 mg/kg body weight to 250 mg/kg body weight, about 0.1 mg/kg body weight to 100 mg/kg body weight, about 1 mg/kg body weight to 50 mg/kg body weight, such as about 0.001 mg/kg/day to about 30 mg/kg/day, about 10 mg/kg/day to about 500 mg/kg/day. In other embodiments, the growth hormone-based prolactin receptor antagonist can be administered in an amount of about 0.005 to about 1 mg/kg/day or in an amount of about 0.01 to about 0.5 mg/kg/day. If administered continuously, the growth hormone-based prolactin receptor antagonist can be administered at a rate known to one of skill in the art, for example, at a rate of about 0.5 μg/kg/hour to about 20 μg/kg/hour, including about 1 μg/kg/hour to about 15 μg/kg/hour, such as about 2 μg/kg/hour to about 10 μg/kg/hour.

Moreover, treatment of a subject with a therapeutically effective amount of a growth hormone-based prolactin receptor antagonist can include a single treatment or can include a series of treatments. In a representative example, a subject is treated with a growth hormone-based prolactin receptor antagonist in an amount ranging between about 0.1 mg/kg body weight to about 5 mg/kg body weight, including about 0.5 mg/kg body weight to about 4 mg/kg body weight, such as about 1 mg/kg body weight to about 3 mg/kg body weight one time per week for between about 1 month to about 24 months, including about 2 months to about 18 months, such as about 2 to about 12 months.

The amount of zinc administered should be based on the amount of elemental zinc present in the preparation, which varies from one capsule to another. For example, a standard capsule of 220 mg of zinc sulfate (ZnSO₄) contains approximately 55 mg of elemental zinc (see, e.g., Al-Gurairi et al, 2002, Brit. J. Dermatol. 146(3):423-431). In another example, a capsule of about 50 mg of zinc gluconate (Zn(C₆H₁₁O₇)₂) contains approximately 7 mg of elemental zinc (see, e.g., Lewis and Kokan, 1998, J. Toxicol. Clin. Toxicol. 36(1-2):99-101).

As compared with other trace metals, zinc is relatively non toxic. Thus, it is possible to administer orally very large amounts of zinc safely, as much as 800 mg/day; normal zinc intake is 10-100 mg/day. Such very large amounts can more than treble blood zinc concentrations (see, e.g., Al-Gurairi et al., 2002, Br. J. of Dermatol. 146(3):423).

Accordingly, in certain embodiments, the methods of the invention provide for administering elemental zinc in an amount of about 0.1 mg/kg/day to about 10 mg/kg/day. Elemental zinc can be administered at about 0.5 mg/kg/day to about 5 mg/kg/day. In other embodiments, elemental zinc can be administered in an amount of about 8 mg to about 800 mg per day, such as about 4 mg to about 400 mg per day.

In embodiments where zinc is administered as oral zinc sulfate, zinc sulfate is administered in an amount of about 0.4 mg/kg/day to about 40 mg/kg/day. In a more representative embodiment, zinc sulfate is administered at about 2 mg/kg/day to about 20 mg/kg/day.

Where food intake by a subject falls and zinc deficiency can occur (e.g., in a subject suffering from cancer cachexia or nausea resulting from chemotherapy or radiation), the amount of zinc administered should be increased in order to counteract decreased zinc intake from diet when administering the growth hormone-based prolactin receptor antagonist.

In embodiments where one or more growth hormone-based prolactin receptor antagonists and zinc are used in a method of preventing a prolactin receptor related condition, e.g., a prolactin receptor related condition of the breast or prostate, in a subject in need of such prevention, the antagonist can be administered in an amount of about 10 μg/kg/day to about 500 μg/kg/day, such as about 15 μg/kg/day to about 100 μg/kg/day, including about 20 μg/kg/day to about 75 μg/kg/day and the zinc can be administered in an amount of about 0.01 mg/kg/day to about 10 mg/kg/day, such as about 0.01 mg/kg/day to about 100 mg/kg/day, including about 0.05 mg/kg/day to about 50 mg/kg/day, including about 0.1 mg/kg/day to about 10 mg/kg/day. Administration of the one or more growth hormone-based prolactin receptor antagonists and zinc can continue indefinitely.

In the methods of the present invention, agents can also be used which modulate expression or activity of the prolactin receptor. Such agents may be identified using, for example, the binding assays of Section 6.2.1.1. and the activity assays of Section 6.2.1.2. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

It is understood that appropriate doses of such small molecule agents depends upon a number of factors known to those or ordinary skill in the art, e.g., a physician. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram.

### 6.7. Pharmaceutical Compositions

Pharmaceutical compositions for use in accordance with the present invention may be formulated in conventional manner using one or more physiologically acceptable carriers or excipients in combination with the growth hormone-based prolactin receptor antagonists and/or zinc.

Thus, the growth hormone-based prolactin receptor antagonists and/or zinc and their physiologically acceptable salts and solvates may be formulated for administration by inhalation or insufflation (either through the mouth or the nose) or oral, buccal, parenteral or rectal administration, as determined to be appropriate by one of skill in the art.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For administration by inhalation, the growth hormone-based prolactin receptor antagonists and/or zinc are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges for use in an inhaler or insufflator may be formulated containing a powder mix of the compound and a suitable powder base such as lactose or starch.

The growth hormone-based prolactin receptor antagonists and/or zinc may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use. In general, water, a suitable oil, saline, aqueous dextrose (glucose), and related sugar solutions and glycols such as propylene glycol or polyethylene glycols are suitable carriers for parenteral solutions. Solutions for parenteral administration contain a water soluble salt of the active ingredient, suitable stabilizing agents and, if necessary, buffer substances. Antioxidizing agents such as sodium bisulfate, sodium sulfite or ascorbic acid, either alone or combined, are suitable stabilizing agents. Citric acid and its salts and sodium ethylenediaminetetraacetic acid (EDTA) may also be included. In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

The growth hormone-based prolactin receptor antagonists and/or zinc may also be formulated in rectal compositions such as suppositories or retention enemas, e.g., containing conventional suppository bases such as cocoa butter or other glycerides.

In addition to the formulations described previously, the growth hormone-based prolactin receptor antagonists may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

In addition, the growth hormone-based prolactin receptor antagonists may be formulated by the attachment of polyethylene glycol, albumin, or some other inert compound to delay the clearance of the compound from the blood as described above.

Additionally, standard pharmaceutical methods can be employed to control the duration of action of the growth hormone-based prolactin receptor antagonists and/or zinc. These are well known in the art and include control release preparations and can include appropriate macromolecules, for example, polymers, polyesters, polyamino acids, polyvinyl, pyrolidone, ethylenevinylacetate, methyl cellulose, carboxymethyl cellulose or protamine sulfate. The concentration of macromolecules as well as the methods of incorporation can be adjusted in order to control release. Additionally, the growth hormone-based prolactin receptor antagonists and/or zinc can be incorporated into particles of polymeric materials such as polyesters, polyamino acids, hydrogels, poly (lactic acid) or ethylenevinylacetate copolymers. In addition to being incorporated, these agents can also be used to trap the compound in microcapsules.

Sustained and/or timed release formulations may also be made by sustained release means or delivery devices that are well known to those of ordinary skill in the art, such as those described in U.S. Pat. Nos. 3,845,770; 3,916,899; 3,536,809; 3,598,123; 4,008,719; 4,710,384; 5,674,533; 5,059,595; 5,591,767; 5,120,548; 5,073,543; 5,639,476; 5,354,556; and 5,733,566, the disclosures of which are each incorporated herein by reference. The pharmaceutical compositions of the present invention can be used to provide slow or sustained release of one or more of the active ingredients using, for example, hydropropylmethyl cellulose, other polymer matrices, gels, permeable membranes, osmotic systems, multilayer coatings, microparticles, liposomes, microspheres, or the like, or a combination thereof to provide the desired release profile in varying proportions. Suitable sustained release formulations known to those of ordinary skill in the art, including those described herein, may be readily selected for use with the pharmaceutical compositions of the invention. Thus, single unit dosage forms suitable for oral administration, such as, but not limited to, tablets, capsules, gelcaps, caplets, powders, and the like, that are adapted for sustained release are encompassed by the present invention.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

Useful pharmaceutical dosage forms, for administration of the compounds of this invention can be illustrated as follows:

Capsules: Capsules are prepared by filling standard two-piece hard gelatin capsulates each with the desired amount of powdered active ingredient, 175 milligrams of lactose, 24 milligrams of talc and 6 milligrams magnesium stearate.

Soft Gelatin Capsules: A mixture of active ingredient in soybean oil is prepared and injected by means of a positive displacement pump into gelatin to form soft gelatin capsules containing the desired amount of the active ingredient. The capsules are then washed and dried.

Tablets: Tablets are prepared by conventional procedures so that the dosage unit is the desired amount of active ingredient. 0.2 milligrams of colloidal silicon dioxide, 5 milligrams of magnesium stearate, 275 milligrams of microcrystalline cellulose, 11 milligrams of cornstarch and 98.8 milligrams of lactose. Appropriate coatings may be applied to increase palatability or to delay absorption.

Injectable: A parenteral composition suitable for administration by injection is prepared by stirring 1.5% by weight of active ingredients in 10% by volume propylene glycol and water. The solution is made isotonic with sodium chloride and sterilized.

Suspension: An aqueous suspension is prepared for oral administration so that each 5 millimeters contain 100 milligrams of finely divided active ingredient, 200 milligrams of sodium carboxymethyl cellulose, 5 milligrams of sodium benzoate, 1.0 grams of sorbitol solution U.S.P. and 0.025 millimeters of vanillin.

Accordingly, the present invention also provides a method of transferring a therapeutic gene to a host, which comprises administering the vector of the present invention, as part of a composition, using any of the aforementioned routes of administration or alternative routes known to those skilled in the art and appropriate for a particular application. The “effective amount” of the composition is such as to produce the desired effect in a host which can be monitored using several end-points known to those skilled in the art. Effective gene transfer of a vector to a host cell in accordance with the present invention to a host cell can be monitored in terms of a therapeutic effect (e.g. alleviation of some symptom associated with the particular disease being treated) or, further, by evidence of the transferred gene or expression of the gene within the host (e.g., using the polymerase chain reaction in conjunction with sequencing, Northern or Southern hybridizations, or transcription assays to detect the nucleic acid in host cells, or using immunoblot analysis, antibody-mediated detection, mRNA or protein half-life studies, or particularized assays to detect protein or polypeptide encoded by the transferred nucleic acid, or impacted in level or function due to such transfer). Where appropriate, the gene therapy vectors can be formulated into preparations in solid, semisolid, liquid or gaseous forms such as tablets, capsules, powders, granules, ointments, solutions, suppositories, injections, inhalants, and aerosols, in the usual ways for their respective route of administration. Means known in the art can be utilized to prevent release and absorption of the composition until it reaches the target organ or to ensure timed-release of the composition. A pharmaceutically acceptable form should be employed which does not ineffectuate the compositions of the present invention. In pharmaceutical dosage forms, the compositions can be used alone or in appropriate association, as well as in combination, with other pharmaceutically active compounds.

Furthermore, the actual dose and schedule can vary depending on whether the compositions are administered in combination with other pharmaceutical compositions, or depending on interindividual differences in pharmacokinetics, drug disposition, and metabolism. Similarly, amounts can vary in in vitro applications depending on the particular cell line utilized (e.g., based on the number of adenoviral receptors present on the cell surface, or the ability of the particular vector employed for gene transfer to replicate in that cell line). Furthermore, the amount of vector to be added per cell will likely vary with the length and stability of the therapeutic gene inserted in the vector, as well as also the nature of the sequence, and is particularly a parameter which needs to be determined empirically, and can be altered due to factors not inherent to the methods of the present invention (for instance, the cost associated with synthesis). One skilled in the art can easily make any necessary adjustments in accordance with the exigencies of the particular situation.

These methods described herein are by no means all-inclusive, and further methods to suit the specific application will be apparent to the ordinary skilled artisan. Moreover, the effective amount of the compositions can be further approximated through analogy to compounds known to exert the desired effect.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### 7. EXAMPLE: TREATMENT OF BREAST CANCER

This example demonstrates the ameliorating effect of a prolactin receptor antagonist plus zinc on reducing the effect of local prolactin excess, or local expression of the prolactin receptor, especially the effect of local prolactin or growth hormone expression or local expression of a factor that stimulates the prolactin receptor or by local prolactin receptor activation.

More than 180,000 women are diagnosed with breast cancer each year in the United States. Treatment efforts are focusing on preventive and early treatment options. According to the ODAC, 40% of breast cancer subjects are candidates for adjuvant chemotherapy (drug therapy in addition to other treatment). The use of a prolactin receptor antagonist plus zinc is one such drug therapy treatment.

If a breast abnormality is detected with mammography or physical exam, a woman will typically be referred for additional breast imaging with diagnostic mammography, ultrasound, or other imaging tests. Depending on the results of these imaging tests, she may be referred for a breast biopsy. Biopsy is the only definitive way to determine whether cancer is present. A pathologist examines the histopathology of the biopsy to determine if cancerous tissue is present. It is now also common practice to examine if hormone receptors are present. For example whether estrogen receptors (ER-positive), or progesterone receptors (PR-positive) are present. This gives valuable information to subjects as cancers which ER or PR-positive receptors tend to have a better prognosis than cancers that do not have these receptors. Cancers with ER or PR-positive receptors are also much more likely to respond to appropriate hormone treatment. In comparison, the importance of prolactin or prolactin receptor in breast cancer is not well recognized. For example, Giordano et al, states that 81% of tumors were estrogen receptor positive, 74% were progesterone receptor positive, 37% overexpressed c-erbB-2, 30% overexpressed p53, 79% overexpressed Bcl-2, 51% overexpressed cyclin D1, and 39% overexpressed epidermal growth factor receptor (see, e.g., Giordano et al., 2002, Ann Intern Med 137(8):678-87). Significantly there was no analysis of prolactin or prolactin receptor expression by Giordano et al.

Biopsies from the subjects are then screened using a test for expression of the prolactin receptor, prolactin, or growth hormone. Such a test is similar to that used to detect HER-2 over-expression in breast tissue (see, e.g., Gorda et al., 2002 J Urol 168(4 Pt 1):1412-4). The HER-2 test uses immunohistochemical staining and can be performed using the HercepTest kit (Dako Corp., Carpinteria, Calif.). In addition prolactin receptor gene amplification methods can be used to detect receptor overexpresssion. These and other methodology can be used to measure the prolactin receptor to discover subjects overexpressing the prolactin receptor.

Following being diagnosed with breast cancer most women undergo treatment for the disease which will depend on many factors including the size and site of the tumor, the stage of the cancer, and the results of lab tests (such as a test for the level of a prolactin receptor expression). Most women with breast cancer will have surgery, either lumpectomy or mastectomy, and in addition some will receive adjuvant (additional) treatment to stop cancer growth, spread, or recurrence. Types of adjuvant therapies include: chemotherapy, radiation therapy and treatment with drugs. Substantial progress in the last few years has been made in the treatment of invasive breast cancer. For the first time, breast cancer mortality rates in the US are decreasing, with refinements in adjuvant treatment contributing to this advance. However, there remains a need for an effective cure.

A variety of drugs may be used with or without breast surgery to treat breast cancer. The most common breast cancer drug, is tamoxifen which is an estrogen receptor antagonist. By blocking estrogen receptors in the breast, tamoxifen helps slow the growth and multiplication of breast cancer cells. Other drugs used to treat breast cancer may include Ellence® which is used in conjunction with chemotherapy (cyclophosphamide and fluorouracil) to treat early stage breast cancer, Taxol® which is used to treat both early and advanced breast cancer, Aromasin® in post-menopausal subjects with advanced breast cancer, Docetaxel® which used in conjunction with chemotherapy to treat advanced breast cancer and Herceptin® which is used in subjects with an excess of the HER-2 receptor. Adjuvant tamoxifen therapy remains the standard therapy for women with estrogen receptor-positive breast cancer (see, e.g., Winer et al., 2002, J Clin Oncol, 20(15):3317-27).

Subjects with breast cancer, as defined above, are administered injections of a growth hormone-based prolactin receptor antagonist plus active zinc supplementation. The amount of the growth hormone-based prolactin receptor antagonist administered is from 10 to 500 micrograms/kg/day and is continued for from 2 to 12 months, or until a therapeutic effect is observed.

For growth hormone-based prolactin receptor antagonists that bind to both the growth hormone and prolactin receptors, dosing is titrated initially using surrogate markers of efficacy, such as serum IGF-1 as markers that a therapeutic drug level has been reached and therefore that there will likely be therapeutic benefit. Blood is obtained at baseline and during treatment, and stored frozen. Total serum IGF-I, free IGF-I, IGFBP-3 and the acid labile sub-unit are assessed by radioimmunoassay or by other equivalent assays which measure these IGF related markers of growth hormone receptor activity. Other markers of a suppression of growth hormone activity can also be used to measure growth hormone receptor activity. Zinc levels in blood are also measured and the amount of zinc titrated to maintain zinc levels at the top of normal range or when zinc related side effects occur.

The goal of therapy is to initially reduce markers of activity of the growth hormone axis by an amount that is considered significant, for example a reduction in IGF-1 levels of 50% should be seen as significant. Efficacy endpoints of the study are objective measures of tumor response. This is as assessed by clinical measures of tumor burden (progression, objectively stable, partial response and complete response) or survival.

### 8. EXAMPLE: TREATMENT OF PROSTATE DISEASES

### 8.1. Benign Prostatic Hyperplasia (BPH)

In the United States, an estimated 9 million men suffer from BPH symptoms which in severe BPH can lead to urinary retention and renal damage. BPH symptoms initially include urinary urgency, increased urinary frequency, and nighttime urination. The usual cause of BPH is hyperplasia within the prostate which leads to an increase in the size of the prostate mostly due to an increase in the number of smooth muscle cells in the stroma. This increase in prostate size leads to urethral obstruction, due to the urethra anatomically passing through the prostate. In addition to an increase in the number of smooth muscle cells there is an increase in muscle tone of these cells. Drugs such as the alpha-1 receptor blockers doxazosin (marketed as Cardura®), terazosin (marketed as Hytrin®), and tamsulosin (marketed as Flomax®) relax the smooth muscle of the prostate and bladder neck to improve urine flow and to reduce bladder outlet obstruction and therefore decrease the severity of BPH symptoms. Because these drugs affect smooth muscle tone they can have dangerous hypotensive side effects in some patients.

BPH is also treated with drugs that affect steroid metabolism, for example Finasteride™ is a synthetic compound that is a specific inhibitor of the intracellular enzyme which converts the androgen testosterone to 5-alpha-dihydrotestosterone (DHT). Because the prostate is an androgen sensitive tissue, Finasteride can reduce prostate size and reduce BPH symptoms. However because it affects androgen metabolism Finasteride can have adverse effects such as impotence, decreased libido and decreased volume of ejaculate.

BPH can lead to serious problems, including a sudden inability to pass urine (acute urinary retention, or AUR). AUR is an emergency condition and is a significant and painful problem that initially must be managed through bladder catheterization (most often taking place in the emergency room), and may ultimately result in BPH-related surgery. In the US about 400,000 men have surgery each year to remove some of the enlarged prostate gland so as to improve the flow of urine through the urethra. If hyperplasia of the prostate is treated by surgery it can result in impotence, urinary problems, and other adverse side effects.

### 8.1.1. Initial Evaluation of Patients

The initial evaluation and work-up is straightforward. It involves a history and physical examination which includes a digital rectal examination (DRE), neurological evaluation, urinalysis, a serum creatinine (to test renal function) and prostate-specific antigen (PSA) tests. A medical history is then obtained which focuses on the genital and urinary symptoms, previous surgical procedures, and sexual function history.

A symptom score questionnaire, such as the American Urological Association Symptom Index (AUA-SI), which is designed to help evaluate symptoms, is then used to determine symptom severity. This is a simple, self-administered questionnaire that addresses several aspects of a patient's voiding function to create a symptom index. A score of 0 to 7 is categorized as mild, 8 to 19 as moderate, and 20 to 35 is categorized as severe. The physician then evaluates the responses and discusses the impact of the patient's prostatic symptoms on quality of life.

A Digital Rectal Exam (DRE) helps the physician establish prostate size and consistency and is an essential part of identifying and excluding patients with prostate cancer. The DRE also helps the physician evaluate neurologic problems that may be the cause of the BPH symptoms. Prostate-specific antigen (PSA) testing helps increase the detection of prostate cancer over DRE alone and many prostate cancers are discovered despite a normal exam and an abnormal PSA result. PSA can also be elevated due to conditions other than prostate cancer. Direct measurement of prostate volume by trans-rectal ultrasound (TRUS) or MRI also helps establish the presence of BPH.

In summary, the doctor examines the prostate digitally, obtains appropriate laboratory tests (urinalysis, creatinine, and PSA), and may administer a symptom questionnaire. Once other diseases are ruled out, the doctor then determines whether the patient is at risk of adverse BPH-related outcomes. Then, the doctor and patient discuss appropriate treatments including treatment with a prolactin receptor antagonist combined with zinc supplementation.

Subjects with BPH, as defined above, are treated with injections of a prolactin antagonist plus zinc active supplementation. The dosage of the prolactin antagonist is from 10 to 500 micrograms/kg/day and is continued for from 2 to 12 months, or until a therapeutic effect is observed.

Dosing is titrated initially using surrogate markers of efficacy, such as serum IGF-1 as markers of therapeutic drug levels and therefore therapeutic benefit. Blood is obtained at baseline and during treatment, and stored frozen. Total serum IGF-I, free IGF-I, IGFBP-3 and the acid labile sub-unit are assessed by radioimmunoassay or other equivalent assays of thse markers of GH receptor activity. Other markers of suppression of GH activity can also be used to measure GH receptor activity. Zinc levels in blood are also measured and the dose of zinc titrated to maintain zinc levels at the top of normal range or at maximally tolerated blood levels or when zinc related side effects occur.

The goal of therapy should be to initially reduce markers of activity of the GH axis by an amount that is considered significant, for example a reduction in IGF-1 levels of 50% is significant. The treatment regimen described herein as utilized in men with symptomatic benign prostatic hyperplasia (BPH) and an enlarged prostate is designed to improve symptoms, including a significant and sustained increase in maximum urinary flow rate; reduce the risk of acute urinary retention; decrease prostate volume as measured by trans-rectal ultrasound (TRUS) or MRI; reduce the probability that surgery and/or prostatectomy will be necessary; and/or slow the progression of BPH.

### 8.2. Prostate Cancer

Prostate cancer affects one in nine men over the age of 65 and represents the most frequently diagnosed cancer in American men. Early detection through testing for prostate specific antigen (PSA) and improved methods of therapeutic or surgical intervention and radiation therapy have greatly reduced the number of fatalities. The most common current therapy for advanced prostate cancer is androgen-ablation, which results in tumor regression over the short-term due to massive apoptosis of androgen-dependent carcinoma cells. In most cases, however, such treatment ultimately results in the recurrence of highly aggressive and metastatic prostate cancer that is androgen independent. In a recent study of 409 subjects with progressive metastatic prostate cancer, and who had also been subject to castration, the median survival of the entire group was 15.8 months (range, 0.9 to 77.8 months); 87% had died at the end of the study (see, e.g., Smaletz et al., 2002, J Clin Oncol 20(19):3972-82).

Many prostate cancer subjects are now initially discovered by prostate-specific antigen (PSA) screening. The initial evaluation and work-up of subjects with prostate cancer is straightforward. It involves a history and physical examination which includes a digital rectal examination (DRE), neurological evaluation, urinalysis, a serum creatinine and PSA tests. A medical history is then obtained.

A DRE helps a doctor establish prostate size and consistency and is an essential part of positively identifying subjects with prostate cancer. PSA tests have helped increase the detection of prostate cancer, compared to DRE alone. Many prostate cancers are discovered after a normal DRE exam and an abnormal PSA result. In subjects with high PSA levels (greater than or equal to 20 ng/ml) a transrectal prostate biopsy has a low complication rate and is relatively well tolerated and histological analysis of the biopsy is used to confirm the presence of cancer (see, e.g., Gerstenbluth et al., 2002, J Urol 168(5):1990-3).

Biopsies from the subjects are then screened using a test for expression of the prolactin receptor. Such a test is similar to that used to detect Her-2 over-expression in breast or prostate tissue (see, e.g., Gorda et al., 2002, J Urol 168(4 Pt 1):1412-4). The Her-2 test uses immunohistochemical staining and can be performed using the HercepTest kit (Dako Corp., Carpinteria, Calif.). A similar test can be used to measure the prolactin receptor to discover subjects overexpressing the prolactin receptor. Subjects that exhibit such overexpression are candidates for treatment with a prolactin receptor antagonist plus zinc.

Subjects with prostate cancer, as defined above, are administered injections of a prolactin antagonist plus zinc active supplementation. The dosage of the prolactin antagonist is from 10 to 500 micrograms/kg/day and is continued for from 2 to 12 months, or until a therapeutic effect is observed.

For growth hormone-based prolactin receptor antagonists that bind to both the growth hormone and prolactin receptors, dosing is titrated initially using surrogate markers of efficacy, such as serum IGF-1 as markers that a therapeutic drug level has been reached and therefore that there will likely be therapeutic benefit. Blood is obtained at baseline and during treatment, and stored frozen. Total serum IGF-I, free IGF-I, IGFBP-3 and the acid labile sub-unit are assessed by radioimmunoassay or by other equivalent assays which measure these IGF related markers of growth hormone receptor activity. Other markers of a suppression of growth hormone activity can also be used to measure growth hormone receptor activity. Zinc levels in blood are also measured and the dose of zinc titrated to maintain zinc levels at the top of normal range or when zinc related side effects occur.

The goal of therapy should be to initially reduce markers of activity of the growth hormone axis by an amount that is considered significant, for example a reduction in blood IGF-1 levels of 50% should be seen as significant. Tumor response is assessed by clinical measures of tumor burden (progression, objectively stable, partial response and complete response) and measures of changes in prostatic involvement and prostate-specific antigen (PSA).

### 9. EXAMPLE: TREATMENT OF HYPERPROLACTINEMIA

Hyperprolactinemia is the most common endocrine disorder of the hypothalamic-pituitary axis. It occurs mostly in women and is detected most commonly due to the subjects complaining of amenorrhea (lack of estrus cycles) and galactorrhea (breast enlargement and milk secretion). The causes of hyperprolactinemia include pharmacological and pathological influences with; pituitary adenomas being a common pathology. In women hyperprolactinemia is associated with decreased libido, infertility, oligomenorrhea/amenorrhea and galactorrhea. In men the disease can cause decreased libido, infertility, gynecomastia and impotence (see, e.g., Luciano, 1999, J Reprod Med 44(12 Suppl):1085-90). Besides the effects of hyperprolactinemia on fertility and sexual dysfunction, there are also effects on bone mineral density, cardiovascular disease, and changes in psychopathology (see, e.g., Meaney, 2002, Life Sci. 71(9):979-92).

Most subjects are treated with dopamine agonist drugs. Surgery is reserved for a few subjects with rare tumors that do not respond to drugs or for subjects who cannot tolerate dopamine agonists (see, e.g., Biller, 1999, J Reprod Med 44(12 Suppl):1095-9).

Pituitary surgery for microprolactinomas and macroprolactinomas in hyperprolactinemia has no significant beneficial effect on the clinical course of the disease. In contrast, this surgery has risks, including a mortality of 0.9%. Therefore, dopamine agonists are the primary choice for all prolactin-secreting microadenomas and macroadenomas (see, e.g., Zacur, 1999 J Reprod Med 44(12 Suppl):1127-31).

### 9.1. Initial Evaluation of Subjects

Work up is well established for subjects with the above symptoms who are suspected of hyperprolactinemia. Blood is drawn and a serum prolactin level is obtained. If the initial level is above the normal range a repeat blood sample is drawn in the morning in the fasting state. Medical history and laboratory tests can be used to eliminate the common causes of hyperprolactinemia. These include pregnancy, primary hypothyroidism and drugs (like neuroleptics) that reduce the pituitary effects of dopamine. If such causes of a high prolactin blood level are excluded, the sella turcica is imaged to investigate if there is a prolactin-secreting pituitary adenoma or other lesion. Then, the subjects exhibiting hyperprolactinemia not induced by such common causes represent candidates for treatment by the methods of the present invention.

The amount of the prolactin receptor antagonist is from 10 to 500 micrograms/kg/day and is continued for from 2 to 12 months, or until a therapeutic effect is observed. Dosing is titrated initially using surrogate markers of efficacy, such as serum IGF-1, as markers of therapeutic drug levels and therefore therapeutic benefit. Blood is obtained at baseline and during treatment, and stored frozen. Total serum IGF-I, free IGF-I, IGFBP-3 and the acid labile sub-unit are assessed by radioimmunoassay or other equivalent assays of these markers of growth hormone receptor activity. Other markers of suppression of growth hormone activity can also be used to measure growth hormone receptor activity. The goal of therapy should be to initially reduce markers of activity of the growth hormone axis by an amount that is considered significant, for example a reduction in IGF-1 levels of 50% is significant. Zinc levels in blood are also measured and the amount of zinc titrated to maintain zinc levels at the top of normal range or at maximally tolerated blood levels or when zinc related side effects occur.

All patents and other publications mentioned in the specifications are indicative of the levels of those skilled in the art to which the invention pertains. All patents and other publications are herein incorporated by reference to the same extent as if each individual publication was specifically and individually indicated to be incorporated by reference.

One skilled in the art readily appreciates that the patent invention is well adapted to carry out the objectives and obtain the ends and advantages mentioned as well as those inherent therein. Human growth hormone-based prolactin receptor antagonists, zinc, pharmaceutical compositions, treatments, methods, procedures and techniques described herein are presently representative of the described embodiments and are intended to be exemplary and are not intended as limitations of the scope. Changes therein and other uses will occur to those skilled in the art which are encompassed within the spirit of the invention or defined by the scope of the pending claims.

1. A method of ameliorating a symptom of a prolactin receptor-related condition in a subject in need of such amelioration, comprising: administering to said subject a human growth hormone-based prolactin receptor antagonist and zinc in an amount effective to ameliorate said symptom.

2. A method of treating a prolactin receptor-related condition in a subject in need of such treatment, comprising: administering to said subject a human growth hormone-based prolactin receptor antagonist and zinc in an amount effective to treat such condition.

3. A method of preventing a prolactin receptor-related condition in a subject in need of such prevention, comprising: administering to said subject a human growth hormone-based prolactin receptor antagonist and zinc in an amount effective to treat such condition.

4. The method of claim 1, 2, or 3 wherein the condition is breast cancer.

5. The method of claim 1, 2, or 3 wherein the condition is selected from the group consisting of hyperprolactemia, breast cancer, mammary carcinoma, adenocarcinoma, lobular (small cell) carcinoma, intraductal carcinoma, medullary breast cancer, mucinous breast cancer, tubular breast cancer, papillary breast cancer, Paget's disease, inflammatory breast cancer, and hormone dependent tumors of the breast.

6. The method of claim 1, 2, or 3 wherein the condition is prostate cancer.

7. The method of claim 1, 2, or 3 wherein the condition is selected from the group consisting of benign prostate hyperplasia, adenocarcinoma, leiomyosarcoma, rhabdomyosarcoma, hyperprolactemia, and hormone dependent tumors of the prostate.

8. The method of claim 1, 2, or 3 wherein said antagonist is administered to a tissue with an effective local concentration of zinc.

9. The method of claim 8 wherein the tissue is breast tissue.

10. The method of claim 8 wherein the tissue is prostate tissue.

11.-15. (canceled)

16. The method of claim 1, 2, or 3 wherein said antagonist and zinc are formulated in a sustained release formulation.

17. The method of claim 16 wherein said zinc is administered orally.

18. The method of claim 1, 2, or 3 wherein the zinc is ZnSO₄.

19. The method of claim 4 wherein said antagonist and zinc are administered in combination with chemotherapy, surgery, or radiation.

20. The method of claim 4, wherein said antagonist and zinc are administered in combination with estrogen receptor antagonist or HER-2 receptor antagonist.

21. The method of claim 5 wherein said condition is hyperprolactemia and said antagonist and zinc is administered in combination with a dopamine agonist.

22. The method of claim 6 wherein said antagonist and zinc are administered in combination with radiation, surgery, or an androgen receptor antagonist

23. The method of claim 7 wherein said condition is benign prostate hyperplasia and said antagonist and zinc is administered in combination with an adrenergic receptor antagonist, an adrenergic receptor agonist, or an androgen receptor antagonist

24. The method of claim 7 wherein said condition is hyperprolactemia and said antagonist and zinc is administered in combination with a dopamine agonist.

25. A pharmaceutical composition comprising a human growth hormone-based prolactin receptor antagonist and an effective amount of zinc.


SEQUENCE LISTING (1 sequence)

SEQ ID NO: 1 - protein, 191 aa, Homo sapiens [sequence omitted]